(19) United States
(12) Patent Application Publication
(10) Pub. No.: US 2025/0143602 A1
(43) Pub. Date: May 8, 2025

(54) System, Method and Computer Program Product for Advanced Gait Analysis

(71) Applicant: Celloscope Ltd., Tel Aviv (IL)

(72) Inventors: Tamir ALOUSH, Kiryat Ono (IL); Levi TEITZ, Tel Aviv (IL); Yuval NAVEH, Haifa (IL)

(21) Appl. No.: 18/939,288

(22) Filed: Nov. 6, 2024

Related U.S. Application Data
(60) Provisional application No. 63/612,587, filed on Dec. 20, 2023.
(60) Provisional application No. 63/557,740, filed on Feb. 26, 2024.
(60) Provisional application No. 63/557,747, filed on Feb. 26, 2024.
(60) Provisional application No. 63/557,753, filed on Feb. 26, 2024.
(60) Provisional application No. 63/557,762, filed on Feb. 26, 2024.
(60) Provisional application No. 63/596,479, filed on Nov. 6, 2023.
(60) Provisional application No. 63/557,757, filed on Feb. 26, 2024.

(51) Int. Cl. A61B 5/11; A61B 5/00

(52) U.S. Cl. CPC A61B 5/112; A61B 5/6898; A61B 5/7267; A61B 5/7278; A61B 2562/0219

(57) ABSTRACT

A gait analysis method comprising providing a data flow generated by inertial sensor/s borne by an end-user who is ambulating, to define a data flow which describes the end-user's gait, and providing a hardware processor configured to derive, from the data flow which describes the end-user's gait, data which characterizes the end-user e.g. center of pressure (COP) trajectory data and/or kinematic data and, optionally, estimating validity of using the inertial sensor to estimate kinematic data and/or generating an output indication of the user's kinematics trajectory data only when the validity is over-threshold.

### CROSS-REFERENCE TO RELATED APPLICATIONS

The present application claims benefit of the following provisional applications, the entire contents of each of which being fully incorporated herein by reference: Application No. 63/612,587, filed Dec. 20, 2023; Application No. 63/557,740, filed Feb. 26, 2024; Application No. 63/557,747, filed Feb. 26, 2024; Application No. 63/557,753, filed Feb. 26, 2024; Application No. 63/557,762, filed Feb. 26, 2024; Application No. 63/596,479, filed Feb. 26, 2024.

### FIELD OF THIS DISCLOSURE

The present invention relates generally to computerized analysis of motion, and more particularly to computerized analysis of human motion which receives sensor outputs borne by a human, typically in real time.

### BACKGROUND FOR THIS DISCLOSURE

“Center of pressure” is a known biomechanical term e.g., as described here:
en.wikipedia.org/wiki/Center_of_pressure_(terrestrial_locomotion).

It is known that “during walking, the COP moves anteriorly throughout the step from the heel at the time of heel strike to the contralateral toes at toe-off, making two diagonal COP trajectories like a butterfly-pattern”
(journals.sagepub.com/doi/pdf/10.1177/1071181321651197#:˜:text=The % 20COP%20is % 20defined%20as,trajectories%20like%20a%20butterfly%2Dpattern.)
the following online publication: www.researchgate.net/publication/321016303_Center_of_Mass_Estimation_Using_Motion_Capture_System describes using motion capture to estimate center of mass.

The butterfly diagram is a standard way to represent COP trajectory data aka a COP “cycle”.
innssi.com/wp-content/uploads/S3F/FootSTEPS/References/Berki-V-et-al.-J-Biomech-2015.pdf
   www.hindawi.com/journals/tswj/2022/9483665/
   and https://pubmed.ncbi.nlm.nih.gov/35782907
   are online locations for Kitichai's 2022 study on “Application of Machine Learning to Predict Trajectory of the Center of Pressure (COP) Path of Postural Sway Using a Triaxial Inertial Sensor”.

The following online reference describes center of pressure (COP) measurements made using only one sensor mounted on the foot, e.g., on the toe: Wu, Chao-Che & Chen, Yu-Jung & Hsu, Che-Sheng & Wen, Yu-Tang & Lee, Yun-Ju. (2020). Multiple Inertial Measurement Unit Combination and Location for Center of Pressure Prediction in Gait. Frontiers in Bioengineering and Biotechnology. 8. 566474. 10.3389/fbioe.2020.566474.

This online reference (www.physio-pedia.com/Center_of_Pressure_(COP)-describes that “Biomechanics researchers use force platforms . . . to measure the COP during . . . walking”, using force plates to directly measure COP.

This online reference ntrs.nasa.gov/citations/20210022913 uses MoCap to find COP data.

Example uses of known forward kinematics formulae are described here: https://www.rosroboticslearning.com/forward-kinematics.

This online reference peerj.com/articles/4640/by Claudiane A. Fukuchi, Reginaldo K. Fukuchi, Marcos Duarte published Apr. 24, 2018, describes a “public dataset of overground and treadmill walking kinematics and kinetics in healthy individuals”.

Kinematic data may include displacement and/or orientation of body segments such as the lower leg from knee to angle or upper leg from hip to knee, and/or joint angles and/or spatio-temporal gait parameters, e.g., for the lower body. Motion capture (aka MoCap) labs are known which record such motion and generate kinematic data accordingly. The Xsens MoCap system may, by way of non-limiting example, be used; this is a wearable technology to measure a subject's gait. Manufacturer's instructions may be followed to install the uniform and the different system's parts on the subjects.

However, according to Wikipedia, motion capture may include any process of recording the movement of objects or people, e.g., where a user wears markers near each joint to identify the motion by the positions or angles between the markers. Acoustic, inertial, LED, magnetic or reflective markers, or combinations thereof may then be tracked. Or, a MoCap system may extract a silhouette of a user from a background, then compute the user's joint angles, e.g., by fitting a mathematical model into the silhouette. Or, in optical MoCap, data captured from image sensors may be utilized to triangulate 3D position of a subject between cameras calibrated to provide overlapping projections. Data acquisition may be implemented using markers attached to an ambulating user. However, alternatively, data may be generated by tracking surface features identified dynamically for each user. Such systems may produce data with three degrees of freedom for each marker, and rotational information may be inferred from the relative orientation of three or more markers. Or, hybrid mocap systems are configured for combining inertial sensors with optical sensors to reduce occlusion. Passive optical systems may use markers coated with a retroreflective material to reflect light generated near the camera's lens. The camera's threshold is set to sample only bright reflective markers and not to sample skin and fabric. Active optical systems may triangulate positions by illuminating one LED at a time very quickly, or plural LEDs with software to identify them by their relative positions.

A method for gait analysis on center of pressure excursion based on a pressure-sensitive mat is described here:
www.ncbi.nlm.nih.gov/pmc/articles/PMC7663129/.

Use of Inertial Measurement Units aka IMUs for minimal sensing of gait, e.g., IMUs on the feet, is described in this online reference:
ris.utwente.nl/ws/portalfiles/portal/144894545/08857314.pdf.

The following online reference describes center of pressure (COP) measurements made using only one sensor mounted on the foot, e.g., on the toe: Wu, Chao-Che & Chen, Yu-Jung & Hsu, Che-Sheng & Wen, Yu-Tang & Lee, Yun-Ju. (2020). Multiple Inertial Measurement Unit Combination and Location for Center of Pressure Prediction in Gait. Frontiers in Bioengineering and Biotechnology. 8. 566474. 10.3389/fbioe.2020.566474.

Physiopedia (www.physio-pedia.com/Center_of_Pressure_(COP)) describes that “center of pressure (COP) is a fundamental concept in the study of human movement and balance. When a person is standing or walking, their body exerts a force on the ground, to which the ground responds by exerting an equal and opposite force, called the ground reaction force (GRF). The COP is the point at which the total force (the GRF) acting on a person's foot or feet is concentrated, and it is a crucial factor in maintaining stability and preventing falls.

The trajectory of the COP, commonly known as a stabilogram, during static balance is frequently used to measure postural control. When standing still, the COP is thought to be an indicator of the motor mechanisms involved in maintaining balance by keeping the center of mass (COM) within the base of support. Falls are correlated with the displacement of the COP at the limits of stability, highlighting the value of investigating dynamic balance to assess the risk of falling. Biomechanics researchers use force platforms and other instruments to measure the COP during various activities such as standing, walking, jumping, and other dynamic movements. The COP measurements can provide insights into the mechanics of human movement and can be used to develop models and simulations to improve understanding of biomechanics and optimize performance in various applications such as sports and physical therapy . . . parameters of COP . . . are used to analyze COP data in various fields such as . . . rehabilitation.”

A co-pending patent document entitled “Approximating Motion Capture of Plural Body Portions using a single IMU Device” is available under USPTO Publication number: 20230137198 which was published May 4, 2023.

A co-pending patent document entitled System, Method and Computer Program Product for Detecting a Mobile Phone User's Risky Medical Condition N is available under USPTO Publication number: 20200375544 published Dec. 3, 2020.

A co-pending patent document entitled System, Method and Computer Program Product for Assessment of a User's Gait is available under USPTO Publication number 20200289027, published 17 Sep. 20.

OneStep is an FDA-listed medical app, downloadable from GooglePlay, that uses smartphone motion sensors to provide immediate, clinically-validated feedback on gait inter alia.

The disclosures of all publications and patent documents mentioned above and elsewhere in the specification, and of the publications and patent documents cited therein directly or indirectly, are hereby incorporated herein by reference in their entirety. If the incorporated material is inconsistent with the express disclosure herein, the interpretation is that the express disclosure herein describes certain embodiments, whereas the incorporated material describes other embodiments. Definition/s within the incorporated material may be regarded as one possible definition for the term/s in question.

### SUMMARY OF CERTAIN EMBODIMENTS

Certain embodiments of the present invention seek to provide circuitry typically comprising at least one processor in communication with at least one memory, with instructions stored in such memory executed by the processor to provide functionalities which are described herein in detail. Any functionality described herein may be firmware-implemented or processor-implemented, as appropriate.

Certain embodiments seek to provide a cyclic COP trajectory of an ambulating user, using an IMU sensor borne by the user to generate IMU data from which the cyclic COP trajectory may be learned by a trained machine-learning model which typically has been trained on paired cyclic IMU and cyclic COP data, where the on paired cyclic IMU and cyclic COP data is collected by having other users, each bearing an IMU sensor, ambulate on a pressure mat with multi-sensors.

Certain embodiments seek to derive a typically cyclic COP trajectory by processing the output/s of inertial sensor/s worn by a person who is ambulating.

Certain embodiments use a single sensor in spontaneous settings (the sensor's position and the user's activity are unknown), and predict the COP trajectory of the complete gait cycle when possible.

Certain embodiments seek to provide a system or method for capturing motion of a moving body, the system including: an IMU interface configured to receive IMU measurements from at least one IMU worn on the moving body; and/or a hardware processor configured to derive from the IMU measurements, a motion capture approximation output including a trajectory, for each individual body portion from among B body portions, which describes the individual body portion's motion during a single repetition (aka cycle) of a repetitive motion, thereby to provide a trajectory set including B body portion trajectories (aka “repetitive activity patterns”), wherein the hardware processor typically uses generative adversarial networks to derive the trajectory set from the IMU measurements, the generative adversarial networks typically including a first network trained to determine physical feasibility of at least one candidate body portion trajectory for at least one specific body portion, from among a multiplicity of candidate body portion trajectories for said specific body portion; and/or a second network trained to determine how well at least one candidate body portion trajectory, from among the multiplicity of candidate body portion trajectories, fits the IMU measurements.

Certain embodiments seek to provide a method or computer program product, comprising a non-transitory tangible computer readable medium having computer readable program code embodied therein, said computer readable program code adapted to be executed to implement a method for capturing motion of a moving body, the method including: providing an IMU interface configured to receive IMU measurements from at least one IMU worn on the moving body; and/or deriving, from the IMU measurements, using a hardware processor, a motion capture approximation output including a trajectory, for each individual body portion from among B body portions, which describes the individual body portion's motion during a single repetition (aka cycle) of a repetitive motion, thereby to provide a trajectory set including B body portion trajectories (aka “repetitive activity patterns”). Typically, the hardware processor uses generative adversarial networks to derive the trajectory set from the IMU measurements, the generative adversarial networks typically including a first network trained to determine physical feasibility of at least one candidate body portion trajectory for at least one specific body portion, from among a multiplicity of candidate body portion trajectories for said specific body portion; and/or a second network trained to determine how well at least one candidate body portion trajectory, from among the multiplicity of candidate body portion trajectories, fits the IMU measurements.

According to certain embodiments, e.g. as described herein, a gait cycle is characterized, and stored in memory, as a cyclic representation of gait characteristics generated by combining, e.g., averaging these gait characteristics over plural strides of a given user, such that each gait characteristic (e.g. relative displacement, velocity, various other kinetic and/or kinematic parameters etc.) at time-point t1 within a user's first stride, is combined with the same characteristic at time-point t1 within various subsequent strides taken by the user, and each gait characteristic at time-point t2 within the first stride is combined with the same characteristic at time-point t2 within various subsequent strides of the user, thereby to yield a “characteristic” or archetypical characteristic of this user at each time point along the stride cycle including (but usually not limited to) time points t1 and t2. It turns out that when a given gait characteristic is measured at a given time-point within the stride of a given user, over many strides of the given user, that gait characteristic tends to cluster about a single value for this specific user (and tends to cluster about different values, for other users respectively).

All or any subset of these embodiments may be combined.

It is appreciated that any reference herein to, or recitation of, an operation being performed is, e.g. if the operation is performed at least partly in software, intended to include both an embodiment where the operation is performed in its entirety by a server A, and also to include any type of “outsourcing” or “cloud” embodiments in which the operation, or portions thereof, is or are performed by a remote processor P (or several such), which may be deployed off-shore or “on a cloud”, and an output of the operation is then communicated to, e.g. over a suitable computer network, and used by, server A.

Analogously, the remote processor P may not, itself, perform all of the operations, and, instead, the remote processor P itself may receive output/s of portion/s of the operation from yet another processor/s P′, may be deployed off-shore relative to P, or “on a cloud”, and so forth.

The present invention typically includes at least the following embodiments:
Embodiment 1. A gait analysis method comprising: providing a data flow which may be generated by at least one inertial sensor typically borne by an end-user who is ambulating, thereby to define a data flow which describes the end-user's gait, and/or providing a hardware processor which may be configured to derive, e.g. from the data flow which describes the end-user's gait, data center of pressure (COP) trajectory data characterizing the end-user. The same processor may be used for both purposes.
Embodiment 2. A gait analysis method comprising: providing a data flow which may be generated by at least one typically inertial sensor which may be borne by an end-user who is ambulating, thereby to define a data flow which describes the end-user's gait, and/or providing a hardware processor which may be configured to derive, from the data flow which describes the end-user's gait, kinematic data characterizing the end-user; and/or estimating validity of using the inertial sensor to estimate kinematic data and/or generating an output indication of the user's kinematics trajectory data only when said validity is over-threshold. The same processor may be used for all or any subset of the above purposes.
Embodiment 3. A method according to any of the preceding embodiments wherein the end-user bears a mobile phone having an integral accelerometer and wherein the single sensor comprises the mobile phone's integral accelerometer.
Embodiment 4. A method according to any of the preceding embodiments wherein a machine-learning model resides in the processor and wherein the machine-learning model is trained, using training data including: plural data flows each generated by a single inertial sensor borne by a user, thereby to define plural users, and/or plural labels comprising data e.g. center of pressure (COP) trajectory data regarding the plural users respectively which was collected using multiple sensors to sense each of the users' gaits.
Embodiment 5. A method according to any of the preceding embodiments wherein the center of pressure (COP) trajectory data characterizing the end-user comprises a prototypical center of pressure (COP) trajectory which the end-user's COP approximately follows each time the end-user proceeds through her/his gait cycle.
Embodiment 6. A method according to claim 1 wherein said at least one inertial sensor comprises at least one accelerometer.
Embodiment 7. A method according to claim 1 wherein said at least one inertial sensor comprises hardware within a mobile device e.g., phone.

The mobile devices or phones mentioned in this document may comprise any model of any cellphone distributed by any manufacturer such as, by way of non-limiting example, Apple iphone 14 Pro, Samsung's Galaxy S23 Ultra, OnePlus 11, Google Pixel 7a, Samsung Galaxy A54 (or A14) 5G, Samsung Galaxy Z Fold 5, Motorola Razr+, Google Pixel 7 Pro.
Embodiment 8. A method according to any of the preceding embodiments wherein said at least one inertial sensor comprises a single sensor.
Embodiment 9. A method according to any of the preceding embodiments wherein the trajectory data which the hardware processor is configured to derive, comprises trajectory/ies of lower body joint angles aka joint kinematics data characterizing the end-user.
Embodiment 10. A method according to any of the preceding embodiments wherein the trajectory data which the hardware processor is configured to derive, comprises center of pressure (COP) trajectory data characterizing the end-user.
Embodiment 11. A method according to any of the preceding embodiments wherein the center of pressure (COP) trajectory data characterizing the end-user is presented to at least one user as a butterfly diagram.
Embodiment 12. A method according to any of the preceding embodiments wherein the at least one inertial sensor comprises a single inertial sensor.
Embodiment 13. A gait analysis system comprising: a hardware processor which may be configured for providing a data flow generated by at least one inertial sensor borne by an end-user who is ambulating, thereby to define a data flow which describes the end-user's gait, and/or a hardware processor which may be configured to derive, e.g. from the data flow which describes the end-user's gait, data e.g. center of pressure (COP) trajectory data characterizing the end-user. The same processor may be used for both purposes.

Additional embodiments include:

A system comprising at least one hardware processor/s each configured to carry out all or a subset of the operations of any of the above methods and each typically being in data communication with others.

A computer program product, comprising a non-transitory tangible computer readable medium having computer readable program code embodied therein, said computer readable program code adapted to be executed to implement a gait analysis, the method comprising: providing a data flow generated by at least one inertial sensor borne by an end-user who is ambulating, thereby to define a data flow which describes the end-user's gait, and/or providing a hardware processor configured to derive, from the data flow which describes the end-user's gait, center of pressure (COP) trajectory data characterizing the end-user.

A computer program product, comprising a non-transitory tangible computer readable medium having computer readable program code embodied therein, said computer readable program code adapted to be executed to implement a gait analysis, the method comprising: providing a data flow generated by at least one inertial sensor borne by an end-user who is ambulating, thereby to define a data flow which describes the end-user's gait, and/or providing a hardware processor configured to derive, from the data flow which describes the end-user's gait, kinematic data characterizing the end-user; and/or estimating validity of using the inertial sensor to estimate kinematic data; and/or generating an output indication of the user's kinematics trajectory data only when said validity is over-threshold.

Also provided, excluding signals, is a computer program comprising computer program code means for performing any of the methods shown and described herein when said program is run on at least one computer; and a computer program product, comprising a typically non-transitory computer-usable or-readable medium e.g. non-transitory computer-usable or-readable storage medium, typically tangible, having a computer readable program code embodied therein, said computer readable program code adapted to be executed to implement any or all of the methods shown and described herein. The operations in accordance with the teachings herein may be performed by at least one computer specially constructed for the desired purposes, or a general-purpose computer specially configured for the desired purpose by at least one computer program stored in a typically non-transitory computer readable storage medium. The term “non-transitory” is used herein to exclude transitory, propagating signals or waves, but to otherwise include any volatile or non-volatile computer memory technology suitable to the application.

Any suitable processor/s, display and input means may be used to process, display, e.g., on a computer screen or other computer output device, store, and accept information such as information used by or generated by any of the methods and apparatus shown and described herein; the above processor/s, display and input means including computer programs, in accordance with all or any subset of the embodiments of the present invention. Any or all functionalities of the invention shown and described herein, such as but not limited to operations within flowcharts, may be performed by any one or more of: at least one conventional personal computer processor, workstation or other programmable device or computer or electronic computing device or processor, either general-purpose or specifically constructed, used for processing; a computer display screen and/or printer and/or speaker for displaying; machine-readable memory such as flash drives, optical disks, CDROMs, DVDs, BluRays, magnetic-optical discs or other discs; RAMs, ROMS, EPROMS, EEPROMs, magnetic or optical or other cards, for storing, and keyboard or mouse for accepting. Modules illustrated and described herein may include any one or combination or plurality of: a server, a data processor, a memory/computer storage, a communication interface (wireless (e.g., BLE) or wired (e.g., USB)), a computer program stored in memory/computer storage.

The term “process” as used above is intended to include any type of computation or manipulation or transformation of data represented as physical, e.g. electronic, phenomena which may occur or reside e.g. within registers and/or memories of at least one computer or processor. Use of nouns in singular form is not intended to be limiting; thus the term processor is intended to include a plurality of processing units which may be distributed or remote, the term server is intended to include plural typically interconnected modules running on plural respective servers, and so forth.

The above devices may communicate via any conventional wired or wireless digital communication means, e.g., via a wired or cellular telephone network, or a computer network such as the Internet.

The apparatus of the present invention may include, according to certain embodiments of the invention, machine readable memory containing or otherwise storing, a program of instructions, which, when executed by the machine, implements all or any subset of the apparatus, methods, features, and functionalities of the invention shown and described herein. Alternatively, or in addition, the apparatus of the present invention may include, according to certain embodiments of the invention, a program as above which may be written in any conventional programming language, and optionally a machine for executing the program, such as but not limited to a general-purpose computer which may optionally be configured or activated in accordance with the teachings of the present invention. Any of the teachings incorporated herein may, wherever suitable, operate on signals representative of physical objects or substances.

The embodiments referred to above, and other embodiments, are described in detail in the next section.

Any trademark occurring in the text or drawings is the property of its owner and occurs herein merely to explain or illustrate one example of how an embodiment of the invention may be implemented.

Unless stated otherwise, terms such as, “processing”, “computing”, “estimating”, “selecting”, “ranking”, “grading”, “calculating”, “determining”, “generating”, “reassessing”, “classifying”, “generating”, “producing”, “stereo-matching”, “registering”, “detecting”, “associating”, “superimposing”, “obtaining”, “providing”, “accessing”, “setting” or the like, refer to the action and/or processes of at least one computer/s or computing system/s, or processor/s or similar electronic computing device/s or circuitry, that manipulate and/or transform data which may be represented as physical, such as electronic, quantities, e.g., within the computing system's registers and/or memories, and/or may be provided on-the-fly, into other data which may be similarly represented as physical quantities within the computing system's memories, registers or other such information storage, transmission or display devices or may be provided to external factors e.g. via a suitable data network. The term “computer” should be broadly construed to cover any kind of electronic device with data processing capabilities, including, by way of non-limiting example, personal computers, servers, embedded cores, computing systems, communication devices, processors (e.g., digital signal processors (DSPs), microcontrollers, field programmable gate arrays (FPGAs), application specific integrated circuits (ASICs), etc.) and other electronic computing devices. Any reference to a computer, controller, or processor, is intended to include one or more hardware devices, e.g., chips, which may be co-located or remote from one another. Any controller or processor may, for example, comprise at least one CPU, DSP, FPGA or ASIC, suitably configured in accordance with the logic and functionalities described herein.

Any feature or logic or functionality described herein may be implemented by processor/s or controller/s configured as per the described feature or logic or functionality, even if the processor/s or controller/s are not specifically illustrated for simplicity. The controller or processor may be implemented in hardware, e.g., using one or more Application-Specific Integrated Circuits (ASICs) or Field-Programmable Gate Arrays (FPGAs) or may comprise a microprocessor that runs suitable software, or a combination of hardware and software elements.

The present invention may be described, merely for clarity, in terms of terminology specific to, or references to, particular programming languages, operating systems, browsers, system versions, individual products, protocols and the like. It will be appreciated that this terminology or such reference/s is intended to convey general principles of operation clearly and briefly, by way of example, and is not intended to limit the scope of the invention solely to a particular programming language, operating system, browser, system version, or individual product or protocol. Nonetheless, the disclosure of the standard or other professional literature defining the programming language, operating system, browser, system version, or individual product or protocol in question, is incorporated by reference herein in its entirety.

Elements separately listed herein need not be distinct components, and alternatively may be the same structure. A statement that an element or feature may exist is intended to include (a) embodiments in which the element or feature exists; (b) embodiments in which the element or feature does not exist; and (c) embodiments in which the element or feature exist selectably, e.g., a user may configure or select whether the element or feature does or does not exist.

Any suitable input device, such as but not limited to a sensor, may be used to generate or otherwise provide information received by the apparatus and methods shown and described herein. Any suitable output device or display may be used to display or output information generated by the apparatus and methods shown and described herein. Any suitable processor/s may be employed to compute or generate or route, or otherwise manipulate or process information as described herein and/or to perform functionalities described herein and/or to implement any engine, interface, or other system illustrated or described herein. Any suitable computerized data storage, e.g., computer memory, may be used to store information received by or generated by the systems shown and described herein. Functionalities shown and described herein may be divided between a server computer and a plurality of client computers. These or any other computerized components shown and described herein may communicate between themselves via a suitable computer network.

The system shown and described herein may include user interface/s e.g. as described herein, which may, for example, include all or any subset of: an interactive voice response interface, automated response tool, speech-to-text transcription system, automated digital or electronic interface having interactive visual components, web portal, visual interface loaded as web page/s or screen/s from server/s via communication network/s to a web browser or other application downloaded onto a user's device, automated speech-to-text conversion tool, including a front-end interface portion thereof and back-end logic interacting therewith. Thus, the term user interface or “UI” as used herein includes also the underlying logic which controls the data presented to the user, e.g., by the system display, and receives and processes and/or provides to other modules herein, data entered by a user, e.g., using her or his workstation/device.

### BRIEF DESCRIPTION OF THE DRAWINGS

Certain embodiments of the present invention are illustrated in the following drawings; in the block diagrams, arrows between modules may be implemented as APIs, and any suitable technology may be used for interconnecting functional components or modules illustrated herein in a suitable sequence or order, e.g., via a suitable API/Interface. For example, state of the art tools may be employed, such as but not limited to Apache Thrift and Avro which provide remote call support. Or, a standard communication protocol may be employed, such as but not limited to HTTP or MQTT, and may be combined with a standard data format, such as but not limited to JSON or XML. According to one embodiment, one of the modules may share a secure API with another module. Communication between modules may comply with any customized protocol or customized query language, or may comply with any conventional query language or protocol.

FIGS. 1-11 are simplified flowchart illustrations of methods (in which all or any subset of the operations may be performed) which are useful for performing embodiments of the present invention, including inter alia:

FIGS. 4-7 which illustrate a MoCap-estimation method which may be used to implement operation a10 a30 and a110 in METHOD1 of FIG. 1; and

FIGS. 8-9, 11 which illustrate a COP-estimation method which may be used to implement operation a20 in METHOD1 of FIG. 1.

FIG. 10 is a table presenting aspects of the invention according to certain embodiments.

FIG. 12 is a graph of kinematics cycle distance vs. IMU cycle distance which is useful inter alia in determining a threshold for translating the confidence in FIG. 13 to valid/invalid.

FIG. 13 is a MoCap validation method which may, for example, be used to implement operation a120 in METHOD1 of FIG. 1; all or any subset of the operations may be performed.

Methods and systems included in the scope of the present invention may include any subset or all of the functional blocks shown in the specifically illustrated implementations by way of example, in any suitable order, e.g., as shown. Flows may include all or any subset of the illustrated operations, suitably ordered, e.g., as shown. Tables herein may include all or any subset of the fields and/or records and/or cells and/or rows and/or columns described.

Computational, functional or logical components described and illustrated herein can be implemented in various forms, for example as hardware circuits, such as but not limited to custom VLSI circuits or gate arrays or programmable hardware devices such as but not limited to FPGAs, or as software program code stored on at least one tangible or intangible computer readable medium and executable by at least one processor, or any suitable combination thereof. A specific functional component may be formed by one particular sequence of software code, or by a plurality of such, which collectively act or behave or act as described herein with reference to the functional component in question. For example, the component may be distributed over several code sequences, such as but not limited to objects, procedures, functions, routines, and programs, and may originate from several computer files which typically operate synergistically.

Each functionality or method herein may be implemented in software (e.g. for execution on suitable processing hardware such as a microprocessor or digital signal processor), firmware, hardware (using any conventional hardware technology such as Integrated Circuit technology) or any combination thereof.

Functionality or operations stipulated as being software-implemented may alternatively be wholly or fully implemented by an equivalent hardware or firmware module, and vice-versa. Firmware implementing functionality described herein, if provided, may be held in any suitable memory device, and a suitable processing unit (aka processor) may be configured for executing firmware code. Alternatively, certain embodiments described herein may be implemented partly or exclusively in hardware, in which case all or any subset of the variables, parameters, and computations described herein may be in hardware.

Any module or functionality described herein may comprise a suitably configured hardware component or circuitry. Alternatively or in addition, modules or functionality described herein may be performed by a general purpose computer, or more generally by a suitable microprocessor, configured in accordance with methods shown and described herein, or any suitable subset, in any suitable order, of the operations included in such methods, or in accordance with methods known in the art.

Any logical functionality described herein may be implemented as a real time application, if, and as appropriate, and which may employ any suitable architectural option, such as but not limited to FPGA, ASIC, or DSP, or any suitable combination thereof.

Any hardware component mentioned herein may in fact include either one or more hardware devices, e.g., chips, which may be co-located or remote from one another.

Any method described herein is intended to include, within the scope of the embodiments of the present invention, also any software or computer program performing all or any subset of the method's operations, including a mobile application, platform or operating system, e.g., as stored in a medium, as well as combining the computer program with a hardware device to perform all or any subset of the operations of the method.

Data can be stored on one or more tangible or intangible computer readable media stored at one or more different locations, different network nodes or different storage devices at a single node or location.

It is appreciated that any computer data storage technology, including any type of storage or memory and any type of computer components and recording media that retain digital data used for computing for an interval of time, and any type of information retention technology, may be used to store the various data provided and employed herein. Suitable computer data storage or information retention apparatus may include apparatus which is primary, secondary, tertiary, or off-line; which is of any type or level or amount or category of volatility, differentiation, mutability, accessibility, addressability, capacity, performance and energy use; and which is based on any suitable technologies such as semiconductor, magnetic, optical, paper, and others.

### DETAILED DESCRIPTION OF CERTAIN EMBODIMENTS

A gait analysis method may be performed, e.g., as described below, including providing a data flow generated by at least one inertial sensor borne by an end-user who is ambulating, thereby to define a data flow which describes the end-user's gait, and providing a hardware processor configured to derive, from the data flow which describes the end-user's gait, center of pressure (COP) trajectory data characterizing the end-user.

According to certain embodiments, given an end-user completing gait cycles while bearing a single sensor where the sensor's position and/or activity (running, walking, standing, ascending/descending stairs, other, etc.) are typically unknown, the system and/or method and/or computer program product of the present invention may determine whether the single sensor's outputs enable valid prediction of a COP trajectory for the gait cycles, and may then predict the COP trajectory on at least one occasion on which it is valid to do so, and not predict the COP trajectory on at least one occasion on which it is invalid to do so. Typically, the system and/or method and/or computer program product predict the COP trajectory on all occasions on which it is valid to do so, and do not predict the COP trajectory on any occasion in which it is invalid to do so.

It is appreciated that any suitable method may be employed (e.g., in runtime operation a105 in method 1) for determining the end-user's activity, e.g., the method described in the following co-owned patent document: Assessment Of A User's Gait—US 2020/0289027 incorporated herein by reference in its entirety.

Runtime operation a105 in the method may use an activity and position model as described in co-pending US patent document 2020/0289027 entitled Assessment Of A User's Gait, incorporated herein by reference in its entirety-that inputs an IMU cycle or gait cycle generated by the user's phone, and responsively outputs the user's activity and the bodily position of the phone.

Any method described herein for validity prediction may be employed and/or an alternative method for validity prediction, typically developed by machine-learning (e.g., using a training set based on any method described herein for validity prediction) may be employed.

According to certain embodiments:
i. MoCap prediction uses a smartphone to provide an IMU (inertial measurement unit) cycle, then uses a trained MoCap prediction model to convert the IMU cycle into a kinematics cycle. The kinematics cycle may then be used to characterize gait, e.g., to diagnose health crises or changes in wellbeing and/or to prioritize healthcare and/or provide feedback to the patient and/or therapist on ongoing physiotherapy, etc. and/or
ii. COP prediction uses a smartphone to provide an IMU cycle, then uses a trained COP prediction model to convert the IMU cycle into a COP cycle which is used to characterize gait, e.g., to diagnose health crises or changes in wellbeing and/or to prioritize healthcare and/or provide feedback to the patient and/or therapist on ongoing physiotherapy, etc. and/or
iii. in validation, a trained validation model may be used to convert the kinematics cycle generated in (i) above from an IMU cycle, into forward kinematics, so as to validate functionality ii above (and then ii above may be activated if functionality iii says it is valid to do so).

The term “IMU cycle” aka inertial cycle as used herein is intended to include any repetitive pattern representing a user's repetitive motor action, such as gait, where, typically, the representation stipulates acceleration and/or velocity and/or displacement and/or gyro data, such as but not limited to as spatial (3D) angular velocity and/or spatial (3D) orientation.

Repetitive patterns may include closed cycle graphs representing gait (e.g., (walking, running, ascending and/or descending stairs) and/or non-gait cycles (e.g., squats, jumps, and other repetitive motor actions).

Center of pressure (COP) parameters, collected during gait, are a useful measure of gait ability. For example, center of pressure (COP) during a gait cycle can be indicative of balance capacity, hence is predictive of fall risk. Center of pressure (COP) measurements have been collected using platform or insole systems, or using a pressure-sensitive mat. Postural control can be quantified using aspects of the trajectory of the center of pressure (COP), given that the COP is not stationary throughout the gait cycle. For example, the center of pressure may be in the region of the heel at heel-strike, and then move forward or anteriorly as the gait cycle proceeds, eventually reaching the region of the toes at toe-off. Also, the center of pressure may wobble from side to side if the person is not steady on her or his feet.

A “stabilogram” represents this trajectory, and center of pressure (COP) parameters may be derived from this trajectory, such as but not limited to the COP's displacement, velocity, acceleration, range (e.g. maximum distance between COP and a reference location), mean and standard deviation of the COP's displacement or location over a given window of time, e.g., over a single gait cycle), frequency of outliers, e.g., the number of times within a given window of time, e.g., over a single gait cycle that one of the above parameters moves beyond a predefined threshold defined for that parameter in a given timeframe, total area enclosed by the COP trajectory over a period of time, e.g., over a single gait cycle, power spectral density, e.g., distribution of COP power over different frequencies, and entropy or degree of randomness in COP trajectory.

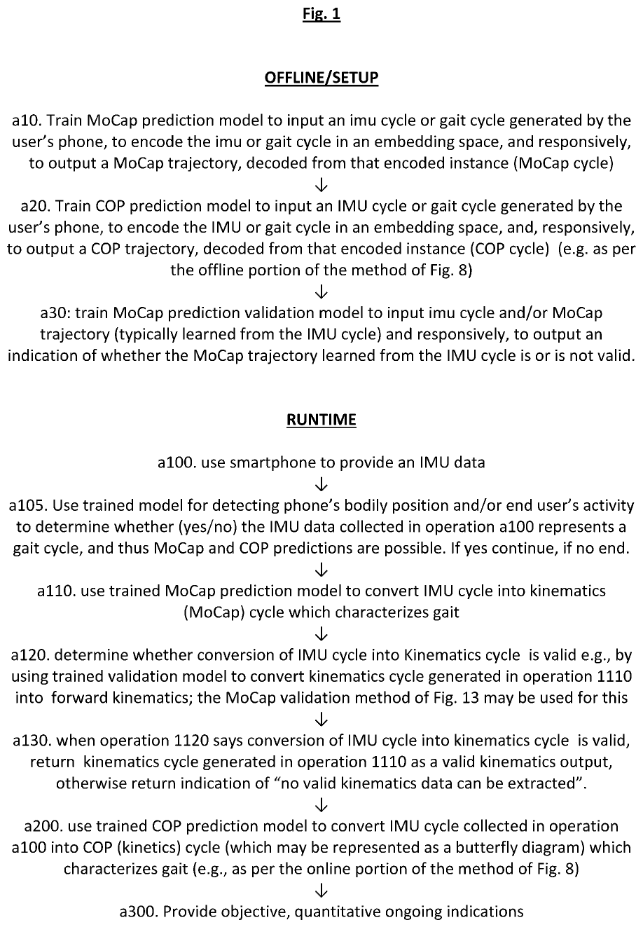

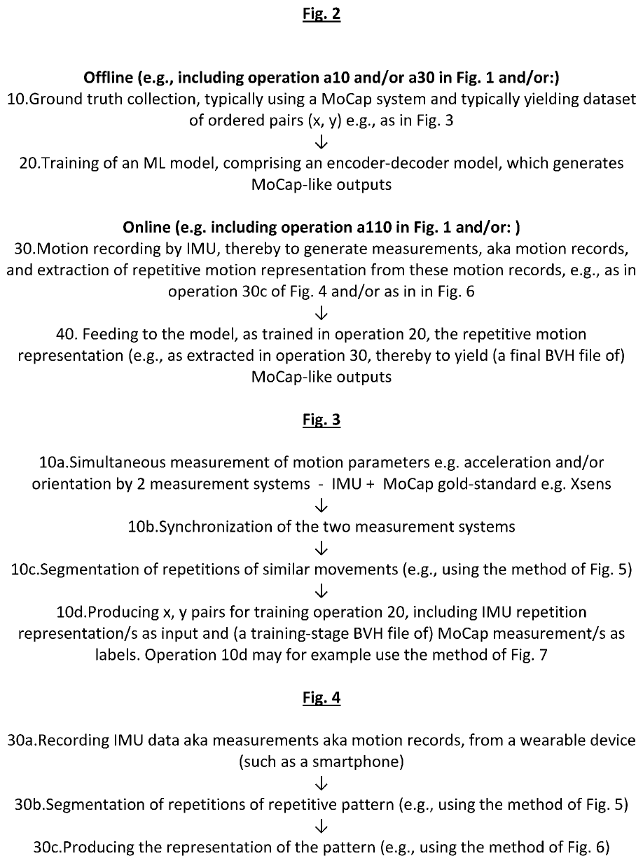

The system of the present invention may comprise a hardware processor/s configured to perform a method (method1) which includes all or any subset of the following operations, suitably ordered, e.g., as shown in FIG. 1:

### OFFLINE/SETUP—Method1: All or Any Subset of

a10. Train MoCap prediction model to input an IMU cycle or gait cycle generated by the user's phone, and, responsively, to output a MoCap trajectory (MoCap cycle) and/or

a20. Train COP prediction model to input an IMU cycle or gait cycle generated by the user's phone, and, responsively, to output a COP trajectory (COP cycle) and/or

a30: train MoCap prediction validation model to input IMU cycle and/or MoCap trajectory (typically learned from the IMU cycle), and, responsively, to output an indication of whether the MoCap trajectory learned from the IMU cycle is or is not valid. Typically, the IMU data is measured or sampled at 100 (say) specific time-points defined along the gait cycle's time-axis, and the kinematic or kinetic data labelling the data during training is also typically measured or sampled at the same 100 (say) specific time-points.

According to certain embodiments, the same number of time-points, e.g. 100 points, are used along each end-user's gait cycle's time-axis, including, say, one end-user whose gait cycle is 1 second long, and another, who, (e.g. due to impairment) has a gait cycle almost 2 seconds long. Thus, the number of points is typically the same over users, whereas the time-interval between points is typically not the same over users. The 100 points are typically evenly spaced along the gait cycle, e.g., for a user whose gait cycle is 1 second long, there may be a fixed time-interval of 0.01 sec between points.

Reference is now made to FIG. 1 which illustrates a method, aka method1, which may be performed according to certain embodiments. All or any subset of the following operations may be performed in method1, in any suitable order, e.g., as follows:

### RUNTIME—Method1: All or Any Subset of

a100. use smartphone to provide IMU data. Any suitable window of data may be collected each time METHOD1 is performed, e.g., using background monitoring as known from “Assessment Of A User's Gait”—co-pending US patent document 2020/0289027 incorporated herein by reference in its entirety or as known from System, Method, and Computer Program Product for Detecting a Mobile Phone User's Risky Medical Condition available under USPTO Publication number: 20200375544, also incorporated herein by reference in its entirety, which also describes active measurement using a mobile app. a105. Use trained model for detecting phone's bodily position and/or end user's activity to determine whether (yes/no) the IMU data collected in operation a100 represents a gait cycle, and thus MoCap and COP predictions are possible. If yes, continue, if no, end.

Typically the method detects whether the data collected is cyclical, hence may be deemed to represent a repetitive activity) and/or recognizes the type of activity (e.g. walking up stairs, running, squats, etc.) and the bodily position in which the phone is deployed e.g. as described in: Assessment Of A User's Gait—co-pending US patent document 2020/0289027 incorporated herein by reference in its entirety. This is done because, typically, the models used in operations a10, a20, a30 herein may be specific to certain bodily positions and/or to certain types of activity or gait.

a110. use trained MoCap prediction model to convert IMU cycle into kinematics (MoCap) cycle which characterizes gait

a120. determine whether conversion of IMU cycle into kinematics cycle is valid, e.g., by using trained validation model to convert kinematics cycle generated in operation 1110 into forward kinematics

a130. when operation 1120 says conversion of IMU cycle into kinematics cycle is valid, return kinematics cycle generated in operation 1110 as a valid kinematics output, otherwise return indication of “no valid kinematics data can be extracted”.

a200. use trained COP prediction model to convert IMU cycle collected in operation a100 into COP (kinetics) cycle (which may be represented as a butterfly diagram) which characterizes gait

a300. Store in memory and/or provide to any suitable end-user via any suitable data communication path to any suitable hardware serving the end-user/s e.g. mobile device/s, typically in real time or in near-real time, objective, quantitative ongoing indications, e.g., change in an individual's gait pattern and/or visualization of same, or improvement/deterioration in recovery, to alert for health crises, e.g., stroke and/or changes in wellbeing and/or fall risk (for seniors e.g.), to prioritize healthcare and/or provide feedback to the patient and/or therapist and/or other caregivers, say on ongoing physiotherapy or other treatment, and/or to a screen, or diagnose malfunctions such as flatfoot, gait asymmetry, etc., based on output indications of changes in (if validated) kinematics cycle and/or COP cycle and/or to justify care necessity. End-users thus may include post-stroke or post-surgery or post-accident users who are doing physiotherapy, users who seek to obtain stroke alerts, or any user or caregiver who participates in a remote care program.

The system may be configured to estimate the COP's displacement, e.g., once per second yielding a COP trajectory. Velocity and acceleration can be derived from this trajectory. Typically, the system is configured to add, to the gait cycle or treadmill known in the art, e.g., by virtue of publication in the following co-owned case: (US 20200289027A1 incorporated herein by reference in its entirety, a learned center of pressure (COP) trajectory over the gait cycle. Typically, a walking/running person has a characteristic COP trajectory that is almost the same for each of his steps; this trajectory may be used as a measure or criterion or tool to assess the person's state, e.g., normality vs. abnormality of the person's gait.

Typically, the system is configured to estimate an average (over plural cycles) deviation of COP values along the gait cycle, from the average COP over the individual's entire gait cycle. Such an average may be estimated separately for each point in the gait cycle. To compute that average for a point half-way along the gait cycle for example, the system may average deviations of the half-way COP value from an average COP over the individual's entire gait cycle, for each of plural cycles.

According to one embodiment, but not necessarily, the system computes the COP trajectory once per step, then averages over plural steps. The model used herein may receive an individual cycle's inertial data, in which case the model generates an individual COP trajectory, or the model may receive inertial data aggregated over plural cycles.

According to certain embodiments, the system-generated COP trajectory generated herein has a known accuracy, for example given users walking on a COP (force plate) system and also carrying a phone in their pocket, thereby to provide data to a system as described herein, the system may generate COP trajectories from the phone's data (typically each comprising an entire gait cycle) which deviate by at most 1 cm from the gold standard COP trajectory generated by the force plate system, for 95% of trajectories so generated.

The system is typically configured to estimate the trajectory of the COP during gait.

This COP trajectory for an ambulating end-user may be estimated using a trained ML model. The labels in the training data may comprise sensor outputs (aka “COP trajectory data”) provided by ground force plate sensor/s and/or pressure sensor/s incorporated into a conventional pressure-sensitive mat. This data may include displacement over time on the ground (e.g., ordinal X, Y pairs, once per unit of time), and magnitude of pressure/force exerted by the user's foot on the mat and/or the data may include (displacement/velocity/accel) of the COP, along all 3 axes—x, y, z—once per (say) second for (say) 15 seconds.

It is appreciated that gait is a complex form of motion, thus it is not necessarily the case, during gait, that the center of pressure is the projection of the center of mass onto the XY plane, because of the dynamics and the moments involved in gait. In contrast, if and when a body is static (but not when a person is walking) the COP does need to apply an equivalent force to that the center of mass applies on the ground.

According to certain embodiments, the position of the sensor is not known (e.g., because the user is “in the wild”, and, in contrast to the laboratory, the user is not constrained to place his sensor in a specific location, such as the user's right pants' pocket or shirt pocket, or strapped to a user's arm or in a user's hand. The system may then first, e.g., in operation a105 of method1 determine the bodily location of the phone (pocket/hand etc.) using any suitable method such as the method described in the following co-owned patent document: Assessment Of A User's Gait—US 2020/0289027 incorporated herein by reference in its entirety.

Typically, this determination is used to determine whether or not to end, depending on whether or not the system finds itself able to provide valid COP/MoCap data.

It is appreciated that validation in real time is valuable because a single sensor can validly provide certain data some of the time, but not all of the time. Validation obviates the necessity of—to date—throwing out good data with bad data. The criteria determining whether or not a single sensor can validly provide certain data is not known in the art. For example, data provided by a single sensor embedded in a mobile phone in a hip pocket of the pants may be valid, whereas the same mobile phone and sensor may generate invalid (“bad”) data if the mobile phone is located in the user's skirt or dress or shirt. And/or, data validity may depend on whether person is running or going up stairs, or just standing. And/or a phone's single sensor may be valid for measuring certain COP trajectory parameters, and not others, e.g., good for one axis of motion, but not for another axis of motion. For example, a phone in a shirt pocket might be able to collect valid data for measuring the anterior-posterior component of the COP trajectory, but data collected by this phone's single sensor regarding lateral components may not be valid.

The system may validate the COP trajectory using MoCap. It is appreciated that, alternatively, or in addition, any other gait cycle parameter may be validated, such as but not limited to spatial gait parameters such as gait speed and/or stride length, temporal gait parameters such as single support phase duration and/or double support duration, and/or COP parameters such as sagittal and coronal sway, or any other function of the COP trajectory or parameter which may be derived from the COP trajectory.

According to certain embodiments, the same hardware (e.g., single sensor), that is capable of validly providing center-of-pressure trajectory estimates only in a subset of cases is, counterintuitively, always (in 100% of cases) capable of validly determining whether or not the sensor's current outputs can validly predict center-of-pressure data. This may occur when generative Al, not having an analytical function, is used (e.g., for providing COP trajectory estimates), whereas forward kinematics, which is analytical and has a closed form, is used for determining validity of using a sensor's current outputs to predict center-of-pressure trajectory data. Validation of this usage of sensor outputs to predict center-of-pressure trajectory data may be achieved, typically in real-time or near-real time by deriving an estimated IMU trajectory from the MoCap, typically in real-time or near-real time, and comparing this estimated trajectory, typically in real-time or near-real time, to the actually measured IMU trajectory (e.g. to the preprocessing output of the IMU data). If the estimated and measured trajectories are similar (e.g., are below a similarity threshold) or matched, the sensor outputs are, typically in real-time or near-real time, deemed valid to predict center-of-pressure trajectory data. If the estimated and measured trajectories are dis-similar (e.g., are above a similarity threshold) or not matched, the system concludes, typically in real-time or near-real time, that the sensor outputs are not valid predictors of center-of-pressure trajectory data. Any suitable similarity criterion or threshold may be employed. For example, the criterion may be that if every joint angle at time t in the estimated trajectory differs by 5 (say) degrees or less from the same joint angle in the measured trajectory, for all time-points t (e.g., throughout the gait cycle), then the 2 trajectories may be considered similar. Otherwise, the 2 trajectories may be considered dissimilar.

It is appreciated that unlike generative Al, which typically lacks an analytical function, forward kinematics is analytical and has a closed form. Forward-kinematics may be employed to determine the location of any portion of a person's foot, e.g. the tip of user's toe at time t, e.g., by computing this location based on known position and orientation of the user, plus known information, which may be derived from single-sensor outputs (e.g. inertial sensor outputs) re motion of joints such as upper leg, lower leg, ankle. For example, to generate a validation model using forward kinematics, an ML model for forward kinematics may be tried, e.g., using the same data described herein, but instead of predicting the MoCap from the inertial representation, the inertial representation is predicted from the MoCap such that the independent and dependent variables are flipped. Typically, since forward kinematics is the same function for every timestamp, pairs of any time index may be used instead of using the complete cycle. e.g., instead of taking pairs of independent variables such as x=100X30 (MoCap) and y=100X6 (IMU), 100 pairs of x=30 (one time index of MoCap) and y=6 (one time index of IMU) may be used for fitting the function.

It is appreciated that training an ML model for forward kinematics aka “implicitly” is not essential, since, alternatively, this function may be defined explicitly or analytically, typically using average (over the user population) human body attributes, e.g., average sizes of segments/body parts. An advantage of training an ML model for forward kinematics is that those body attributes do not need to be defined explicitly, and a “general body model” may be defined as the best-fitting model for the training set data (the body model which best fits the training set data). The body model may be defined in accordance with Wu G, Cavanagh PR. “ISB recommendations for standardization in the reporting of kinematic data.” J Biomech. 1995 Oct.; 28 (10): 1257-61. Doi: 10.1016/0021-9290 (95) 00017-c. PMID: 8550644.

More generally, a suitable method (“method v”) for online validation of MoCap output may include all or any subset of the following operations v1-v5, in any suitable order, e.g., as shown:

V1. MoCap output validation functionality aka validator gets the measured IMU cycle, and the predicted MoCap cycle.

V2. Validator computes the reconstructed IMU cycle from the predicted MoCap cycle using forward kinematics.

V3. Validator compares the measured IMU cycle and the reconstructed IMU cycle using a suitable distance function (e.g. Euclidian or cosine distance) to compute distances between IMU cycles.

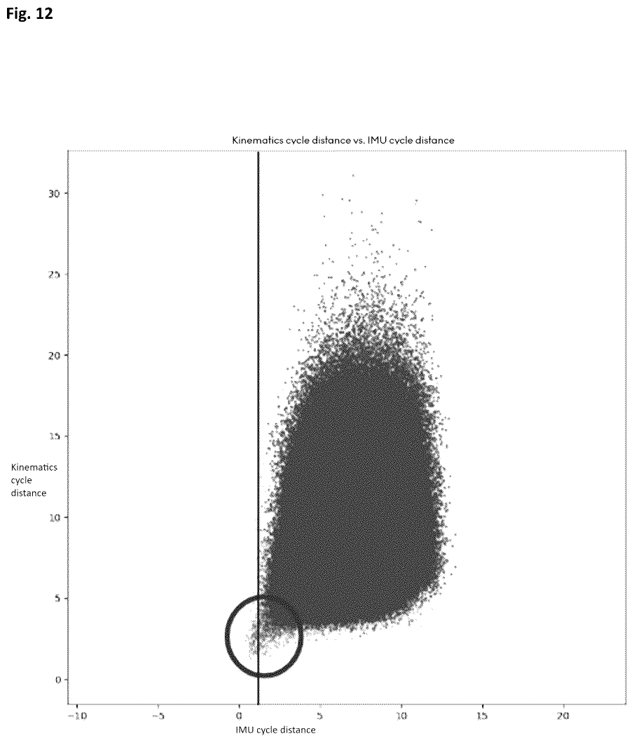

A cyclic Euclidian distance may be computed as the minimum of all Euclidian distances (between two vectors in Euclidian space) between cycle a and cycle b over all rotations of cycle a relative to cycle b, where cycles are defined in a d-dimensional space, where d=k*100 (assuming 100 samples per cycle), and where k is the number of channels as defined in FIG. 10.

A “rotation” of a cycle by r units may be defined by shifting the cycle's values r indexes to the right, where indexes that go beyond the cycle length (100 units) typically go back to beginning of the cycle (e.g. index 107 gets back to index 7).

A correlation distance or cyclic cosine distance may be defined as the minimum of all cosine distances between cycle a and cycle b over all rotations of cycle a relative to cycle b.

A measure of distance between two IMU cycles may be defined as a sum of correlation distances or cyclic Euclidian distances or any combination of them, typically in all 6 channels, between two IMU cycles.

A measure of distance between two COP cycles may be defined as a sum of correlation distances or cyclic Euclidian distances or any combination of them, typically in all 3 channels between two COP cycles.

A measure of distance between two COP cycles may be defined as a sum of correlation distances or cyclic Euclidian distances or any combination of them in all 30 channels between two MoCap cycles.

V4. Validator determines if the two IMU cycles are similar (e.g., are closer than 1 or any pre-defined threshold)

V5. Validator determines that the predicted MoCap is valid (or invalid) if the two IMU cycles are (not) similar.

Thus typically, the MoCap is valid each time the reconstructed IMU trajectory (aka estimated trajectory) is close enough (below a similarity threshold) to an original or measured IMU trajectory.

It is appreciated that all or any subset of the operations of method v may be used to perform operation 2050 of FIG. 13 (feeding to a trained validation model repetitive motion representation (e.g. as extracted in operation 30) and MoCap-like outputs to yield a confidence score of the output validity).

Once the validation model has been generated/trained, this model may be used to derive an estimated IMU trajectory from the MoCap and compare input and output, e.g., compare this estimated trajectory to the IMU trajectory that the system has actually measured (e.g., the pre-processing output of the IMU data).

It turns out that, at least when the IMU is in the pants' pocket, there is a correspondence (typically bijection or one-to-one correspondence) between the following 2 signals:
a. kinematics of the lower body (e.g. 3 channels for the position (x, y, z)+27 channels of joint angles (yaw, pitch, roll): pelvis+r/l hip, knee, ankle over the gait cycle, say 100 units=30*100 matrix) and
b. the trajectory of the IMU (in the pants' pocket and probably in other positions as well) over the gait cycle (3 for position, 3 for orientation=6*100 matrix).

Typically, not all configurations are achievable; instead, only a subset of configurations can be associated with natural/actual walking. This subset typically comprises a subspace which may be projected in either the 30*100 space or the 6*100 space.

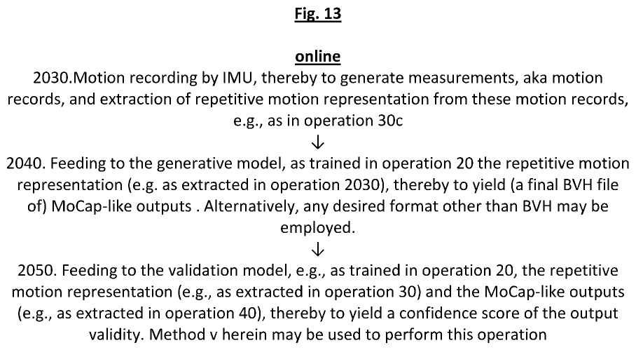

For example, FIG. 12 is a scatter diagram presenting distances between 2 walks, where distance between corresponding kinematics cycles may be computed as described above.

Specifically, the scatter diagram of FIG. 12 represents the kinematics distance (e.g., distance between corresponding kinematics cycles) vs the IMU distance (e.g., distance between corresponding IMU cycles). Between any two samples (two walks), each corresponding to a pair of cycles including a kinematics cycle which may be used as a label for a simultaneously measured aka corresponding IMU cycle (estimated trajectory vs IMU trajectory that was measured typically simultaneously). Thus, typically, walks having similar IMU cycles also have similar kinematic cycles. If the system derives a IMU trajectory from the MoCap data (lower body over time) which is similar to the measured trajectory, this typically means that there could not be any other MoCap data that could be matched with the same IMU trajectory which implies validity.

In the graph of FIG. 12, the x axis is typically the sum over the IMU cycle's channels of the correlation distances which may be used to define IMU distance. The y axis is the average over the kinematics cycle's channels of the Euclidian distances, which may be used to define the kinematics distance.

The vertical line (at IMU cycle distance=1 approximately) in the graph of FIG. 12 separates between pairs with a small IMU distance (e.g., <1) and a high IMU distance (e.g., >1). As is apparent from the graphed results, pairs with small IMU distances imply small kinematics distances. Specifically, the scatter in the graph of FIG. 12 implies that if two IMU cycles are closer than 1 using IMU distance (hence correspond to points to the left of the vertical line) the two IMU cycles will almost always have kinematic distances smaller than 5 (as can be seen inside the left side of the circle marked in FIG. 12).

Typically, validity is defined as a binary variable. Any suitable setup experiments may be done offline, to determine a cutoff point or difference threshold (at least initially), for differentiating valid from invalid e.g., 5 degrees, for all angles.

For example, according to some definition of IMU cycle distance, IMU distance of 1 may be defined as the threshold between the measured IMU cycle and the one produced using forward kinematics on the predicted kinematics. This guarantees that the distance between corresponding kinematic cycles for each of the IMU cycles will be, say, less than 5 kinematic distance units. The sum (over the channels) of the correlation distances is used to operationalize IMU distance and the average (over the channels) of the Euclidian distances is used for kinematic cycles, e.g., as seen in the scatter diagram of FIG. 12, which may be deemed close enough. Hence, if the reconstructed IMU cycle is, say, less than 1 IMU distance units from the original IMU cycle, the kinematics cycle may be deemed valid. It is appreciated that one implementation of IMU distance between two input gait cycles may be the sum of correlations in all 6 channels between the two cycles.

The correlation distance is typically bounded between 0—completely correlated and 2—anti-correlated. The sum of the correlations over, e.g., 6 channels, is thus typically bounded between 0 and 12.

Alternatively, any other cut-off may be defined for the similarity between two kinematics (e.g. 10 degrees at most), the system may then learn the difference threshold between two IMU cycles, e.g., as described above.

Typically, the MoCap is valid if the reconstructed IMU trajectory (aka estimated trajectory) is close enough (below a similarity threshold) to the original or measured IMU trajectory.

Typically, the training data used for the validation comprises P pairs of IMU data and MoCap data segmented into strides, where the MoCap data may be used as labels for the IMU data. Typically, the P pairs include various different users, e.g., P users, rather than a single user who is walking on a pressure mat P times. Also, typically, plural 10 gait variations are included, such as, say, slower gait without limp, faster gait without limp, limping gait.

When the model is being trained, the model tries to derive IMU data from the MoCap data.

It is appreciated that when training a validation model aka validator, the model during training derives IMU data from MoCap data as described above. In contrast, a MoCap prediction model may be trained by deriving MoCap data from IMU data, as described elsewhere herein.

This facilitates implicit (e.g., as described above) learning of a general body model, for example.

The same data pairs may be used with opposite directions of prediction to train a MoCap prediction model in one case (predicting MoCap data from IMU data, e.g., as described elsewhere herein), and a forward kinematics model (predicting IMU data from MoCap data, e.g., as described herein with reference to the scatter diagram of FIG. 12.

It is appreciated that alternatively or in addition, any known forward kinematics formulae may be used to implement the validator, e.g., if certain parameters, such as body segment sizes, are predetermined.

An example method (“method T”) for training, then testing or validating ML models offline is now described; the method may be used to collect data for COP prediction and all or any subset of the following operations j1-j10 may be performed, in any suitable order e.g., as follows:
j1. Ask a first set of 100 (say) users to walk on a pressure sensitive mat
j2. Collect 100 data flows each generated by a single inertial sensor borne by a user from among 100 (say) users (e.g., each user in the first set)
j3. Generate 100 labels comprising center of pressure (COP) trajectory data regarding the gaits of the 100 users, which is derived from sensor outputs of the pressure-sensitive mat
j4. Pair the 100 labels generated in operation j3, with the 100 data flows generated by the 100 users' cellphones respectively in operation j2. Use those 100 (say) labelled data elements as training data to train a COP data-generator which will compute COP data (COP trajectories or COP parameters which may be derived from COP trajectories) from the output flow of users' cellphones.

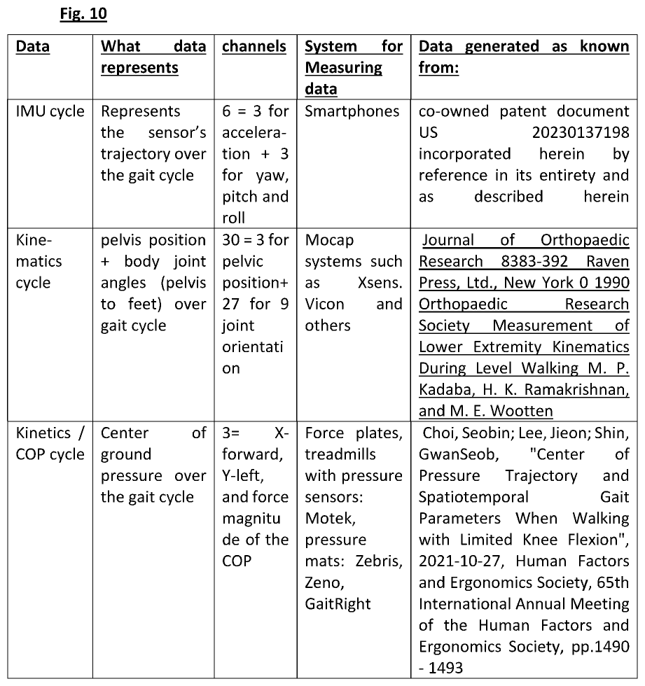

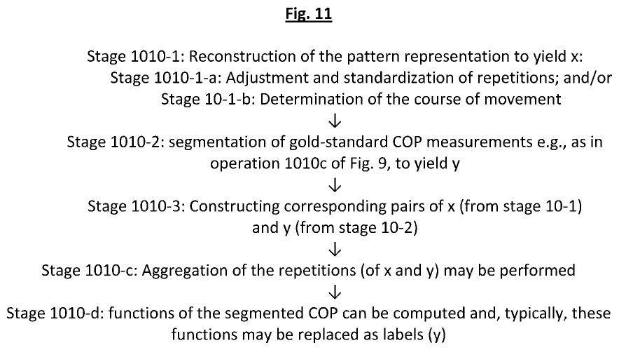

It is appreciated that all or any subset of operations j1-j4 may be replaced by all or any subset of the operations of FIG. 9, mutatis mutandis, and vice versa.
J5. Ask a second set of 101 (say) users to walk on the pressure sensitive mat. This yields sensor outputs both from the 101 users' cellphones' accelerometers, and from the mat's pressure sensors.
J6. Run the trained COP data-generator (as trained in operation j4) on the 101 (say) output flows of the cellphone accelerometers of 101 users respectively. This will yield 101 COP trajectories (or 101 sets of COP parameters which may be derived from COP trajectories).
J7. Generate 101 center of pressure (COP) trajectories (or parameters derived therefrom) regarding the gait of the 101 users, which is derived from sensor outputs of the pressure-sensitive mat
J8. Compare the center of pressure (COP) trajectories (or parameters derived therefrom) generated in operation j6, with the center of pressure (COP) trajectories (or parameters derived therefrom) generated in operation j7, using a suitable distance metric such as the COP standard deviation, e.g., on x or y axis over time, or the ratio between two heel strike forces/pressures. This yields 101 distances between the 2 center of pressure (COP) trajectories (or parameters derived therefrom) for each of the 101 users.
J9. Label each of the 101 (say) center of pressure (COP) trajectories (or parameters derived therefrom) generated in operation j6 as valid or invalid, by thresholding the 101 distances computed in operation j8, such that the trajectories/parameters associated with smaller distances are labelled as valid (COP is valid) and the trajectories/parameters associated with longer distances are labelled as invalid
J10. Compute the percentage of the valid labels out of the 101 (say) labels and determine the percentage of times in which the predictor outputs an accurate COP trajectory.

The operations of the above Method T may, for example, be used to implement or replace all or any operations in the offline portion of the method of FIG. 8 and/or all or any operations in the method of FIG. 9.

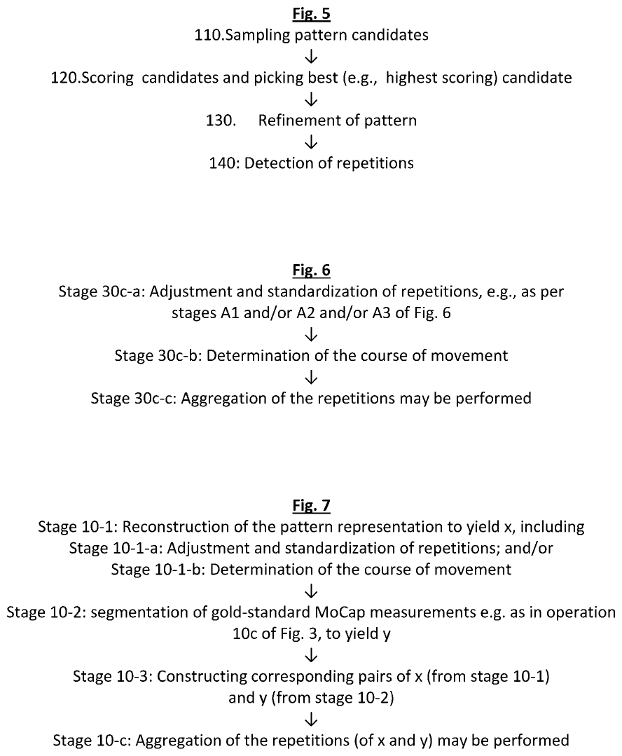

It is appreciated that to collect data for MoCap prediction, FIG. 3 and/or all or any subset of the following operations may be employed, in any suitable order, e.g., as shown:

MoCap 1. Ask a set of 100 (say) users to walk while being recorded simultaneously by an IMU deployed at, say, pants' pocket, and by a motion capture system.

MoCap 2. Collect 100 (say) respective data flows each generated by a single IMU borne by a user from among the 100 (say) users (e.g., each user in the first set)

MoCap 3. Collect 100 (say) labels comprising Motion Capture data regarding the gait of the 100 users, which is derived from the Motion Capture System's outputs.

Referring again to method T, it is appreciated that the above operations j5-j10 may be used for estimating COP prediction, general accuracy, and validity offline.

Typically, the phone measures inertial data and uses a standard representation of the inertial data, e.g., 6*100—the IMU over the gait cycle) and outputs:
1. MoCap cycle (30*100)—kinematics, position+27 joint angles over the gait cycle
2. COP trajectory (3*100)—x, y, | | F| | over the gait cycle.

Offline training of MoCap data-generator to be used for kinematics generation may proceed as described elsewhere herein with reference to FIGS. 8-11 or with reference to operation 20 of FIG. 2.

The following “method k” may be used, typically in runtime, for kinematics generation (for example, method k may be used in implementing the online portion of the method of FIG. 2):

J101. Provide app for phones which includes MoCap-data generation functionality based on the MoCap data-generator as trained offline

J102. User's phone's MoCap-data generation functionality is activated (automatically or by user)

J103. MoCap-data validator at least once, e.g., periodically or constantly, determines whether the MoCap-data generation functionality's output is or is not valid J104. While the most recent output from the MoCap-data validator indicates that the MoCap-data generation functionality's output is valid, the MoCap-data generation functionality provides an output indication of MoCap data (MoCap trajectories or MoCap parameters which may be derived from MoCap trajectories)

J105. Each time the most recent output from the MoCap-data validator indicates that the MoCap-data generation functionality's output is not valid, the MoCap-data generation functionality does not provide any output indication of MoCap data (MoCap trajectories or MoCap parameters which may be derived from MoCap trajectories) and may not generate such data at all, and may instead provide an output indication of invalidity, until the MoCap-data validator indicates that the MoCap-data generation functionality's output is now valid, or until the MoCap-data generation functionality is turned off automatically, or by the user.

In the present application, MoCap data typically includes kinematic data which may be recorded by gold-standard systems for sensing kinematic data such as MoCap systems or may be predicted from IMU data, e.g., as described herein, using a machine learning model which is typically trained on data pairs including IMU data paired (or labelled) with MoCap measurements taken from an ambulating user who is both bearing an IMU sensor (e.g. smartphone) and, simultaneously, being recorded by a MoCap system.

It is appreciated that each MoCap trajectory or cycle is typically cyclic (as are IMU trajectories or cycles and COP trajectories or cycles referred to elsewhere herein, and typically occur over a user's gait cycle, which is repetitive. It is appreciated that MoCap (or IMU) trajectories are not limited to displacement data, and instead may include all or any subset of MoCap channels such as angles and/or orientations.

Regarding COP trajectory prediction (typically without validation), a suitable method for offline training of a COP trajectory prediction model is shown in FIGS. 8-9, and a suitable method for COP trajectory prediction, once the model has been trained, may comprise all or any subset of the following operations k1-k3, suitably ordered e.g. as follows:
k1. Provide app for phones which includes COP-data generation functionality based on the COP data-generator trained offline
k2. User's phone's COP-data generation functionality is activated (automatically or by user)
k3. COP-data generation functionality provides an output indication of COP data (COP trajectories or COP parameters which may be derived from COP trajectories

It is appreciated that trajectories of MoCap/kinematic parameters, such as pelvic tilt, knee internal/external rotation, pelvic obliquity, pelvic rotation, hip flexion/extension, foot int/external rotation, ankle add/abduction, and so forth may be graphed where the x axis of the graph may represent the gait cycle phase and be expressed in % of gait cycle units and the y axis of the graph may represent the joint angle and may be expressed in angle units such as degrees or radians.

FIG. 10 is a table which summarizes aspects of IMU, kinematic (MoCap) and kinetic (COP) cycles where the IMU cycle may comprise an input of the process or of the models of A10 and A20), and the MoCap cycle and/or COP cycle may comprise outputs of the process or of the models trained in operations A10 and A20 of FIG. 1 respectively.

The IMU cycle and the kinematic (MoCap) cycle may comprise the inputs of A30's model.

Regarding the example parameters in the table of FIG. 10, the parameter value 3 represents axes XYZ, 6 represents degrees of freedom (both angular and linear), 30 represents position plus 27 angular positions of 27 lower body joints respectively, and 100 represents a number of IMU samples per gait cycle. These parameters are merely exemplary, e.g., typically, each user's gait cycle is divided into, say, 100 time units such that, e.g., the middle of the gait cycle is 50 time units. However, alternatively, the x-axis of the repetitive cyclic data may be divided into any other number of units.

An example embodiment for a system for capturing kinetic analysis of gait, is now described in detail, which typically includes all or any subset of the following:
an IMU interface configured to receive IMU measurements from at least one IMU worn on the moving body; and/or
a hardware processor configured to derive from the IMU measurements, a motion capture approximation output including kinematic parameters during a single repetition (aka cycle) of a repetitive motion, thereby to provide a kinetic parameters (aka “kinetic analysis”).

The hardware processor typically uses ML networks to derive kinetic parameters from the IMU measurements. The kinetic parameters may include all or any subset of the following:
COP data, e.g., a COP butterfly representation—Fz, which is the total force in the downward direction applied to the treadmill belt at each timepoint; and/or COPx and COPy, the coordinates of the center of pressure on the treadmill belt in the x- and y-axis (the two axes of the transverse plane of the subject); and/or maximum and/or minimum and/or average and/or sums of forces (which may be normalized by subject weight, and/or average applied force) over time at various gait stages, such as single support of each leg and/or double support and/or time surrounding each gait event;
maximum and/or minimum and/or average of deviation of the COPx or/and COPy over time at various gait stages, such as single support of each leg and/or double support, and/or time surrounding each gait event.

Alternatively or in addition, the hardware processor may be configured to derive from the IMU measurements, a motion capture approximation output aka MoCap-like output including a trajectory, for each individual body portion from among B body portions, which describes the individual body portion's motion during a single repetition (aka cycle) of a repetitive motion, thereby to provide a trajectory set including B body portion trajectories (aka “repetitive activity patterns”). The system may be configured for determining if the IMU measurement received could be validly measured from a motion, which is fully or partially described by the set including B body portion trajectories.

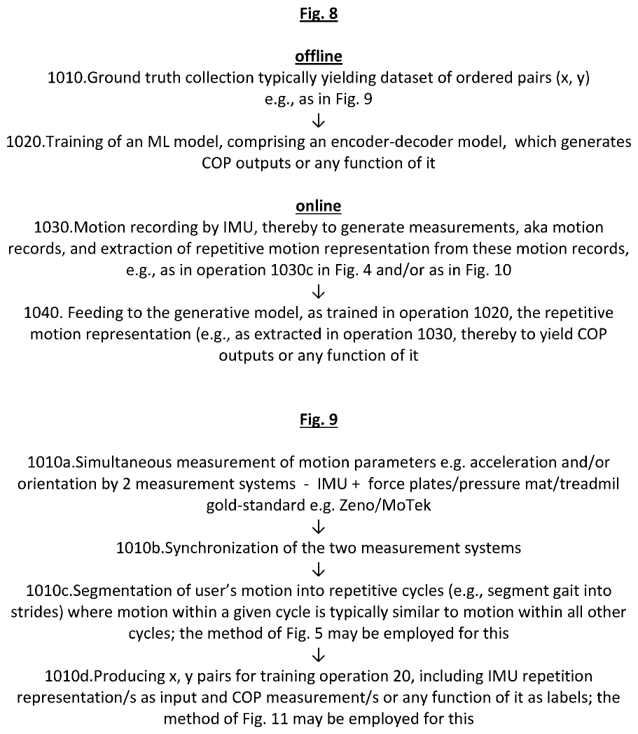

Any or all of the methods of FIGS. 2-11 may be provided, suitably ordered, e.g., as shown. FIGS. 2-7 are useful for generating MoCap predictions from IMU data. These and equivalents are known in the art, e.g., as described in co-owned patent document US 20230137198 entitled “Approximating Motion Capture of Plural Body Portions using a single IMU device” and incorporated herein by reference in its entirety.

FIGS. 8-11 are useful for generating COP predictions from IMU data. FIG. 13 is a simplified flowchart illustration of a method for validation, which determines, typically in real-time or near-real time or online, whether MoCap output, predicted from phone data, is reliable. The MoCap output may be generated in accordance with the methods of FIGS. 2-7.

In FIG. 11 (see e.g. Stage 1010-d in FIG. 11), a deep learning model may be trained for individual kinetic parameters (e.g. maximum loading force during heel strike), typically using the IMU data as input and the associated parameter values as labels (e.g. after splitting the data into training and validation subsets). The model thus created takes IMU data as input, and predicts the parameter value for the specific parameter (e.g., maximum loading force during heel strike) that the model was trained on.

Typically, e.g., in the method of FIG. 13, the MoCap-like outputs comprise kinematic outputs which include MoCap predictions, generated from IMU outputs by a machine learning model trained on MoCap outputs paired with IMU outputs.

Kinetics data may be provided by the system in accordance with the center of pressure analysis. Center of pressure (COP) analysis conventionally refers to motion labs recording the force feet apply on the ground while walking, and is used in medical applications. The system herein may be configured to extract (human) locomotion kinetics representation, COP in particular, from a gait cycle measured by an inertial measurement unit (IMU) placed in a single bodily position. Rather than being restricted to walking, a gait cycle may more generally refer to any repetitive motion activity a subject can perform (such as climbing stairs, running, jumping, etc.).

Offline extraction of (human or other subjects) locomotion representation from a gait cycle may employ a machine learning (ML) scheme, and may include collecting ground truth data for training and validation—typically including operation 1010a which may provide simultaneous measurement of the IMU recording, and external pressure mat or a treadmill with COP measurement capability—gold standard measurement systems, and/or operation 1010b which may include Synchronization of the two measurement systems to provide pairs, each including an IMU repetition representation as input, and a COP measurement as a label.

Typically, COP representations include a standard format e.g., some or all of (x-forward, y-left, and force) in any typically standard order.

Operation 20 comprises training of an ML model, comprising an encoder-decoder model, which may generate COP outputs.

Any suitable representation of COP data or locomotion kinetics data may be provided, such as but not limited to a butterfly diagram. This representation or data may serve as the label component of the pairs produced in operation 1010d of FIG. 9 and may be used to train the model in operation 1020 of FIG. 8. It is appreciated that each COP sample may, for example, store one COP standard representation of locomotion kinetics, which includes the position of the center of pressure in x, y relative to the mean (of each stride) and the course of the walking (=the y axis), and the magnitude of the ground force (in Newtons, or relative to the average force). Typically, in operation 1010d, the pairs each include an IMU repetition representation as input and a COP measurement as a label. The former may include a standard representation of a single repetition as measured by the IMU, and the latter may include a standard representation of a single repetition as measured by pressure mat/treadmill.

Ground truth collection for COP reconstruction typically occurs in operation 1010 of FIG. 8. In this operation, a ground truth dataset is generated, for use in training e.g., as a training set, and/or for validation, including determining the accuracy of models. If dataset of n samples= (IMU,COP)ⁿ, then, according to any embodiment, an IMU repetitive pattern representation/an IMU segment=IMU and/or a center of pressure segment=COP (or any function of it). It is appreciated that IMU may be plural IMU measurements, or one such measurement. Typically, the ground truth dataset includes (IMU, COP)ⁿ pairs where each IMU is an IMU repetitive pattern representation or IMU segment, and each y is a COP segment or a segment generated by any other gold-standard pressure mat system, typically in x, y center of ground force position over time, and the magnitude of force.

The ground truth dataset typically comprises plural samples of repetitive motion representation (which may be generated by segmentation operation 30b) as input and their corresponding COP outputs (repetitive human locomotion COP samples which may be generated by segmentation operation 10c) as labels. Each label typically includes COP sample corresponding to the time interval of the IMU segments. The ground truth is typically a representative sample of the reality, and may challenge the ML models as much as the reality may challenge their accuracy. It is recommended that the ground truth will include hundreds of subjects, a good representation of the general population, performing various kinds of walks by different instructions (asymmetrical walks with different step lengths for the right and the left leg, imitations of various limitations such as walking on the toes on one leg, or on the heels only, or not bending the knee, etc.), known as the measurement protocol. Each portion of a specific walk in which a subject performed a given instruction may include plural, e.g., at least 20 repetitions, before the subject is given a different instruction, and each subject's movement may be measured from different bodily positions. In total, the ground truth sample may include (say) hundreds of thousands, or more, pairs of repetitive motion representations and labels, where both members of each pair are typically generated by segmentation; it is appreciated that segmentation of IMU data (say) into strides or gait cycles is described inter alia in the following co-owned patent documents:
Assessment Of A User's Gait—US 2020/0289027; and
Efficient System Configured To Facilitate Physical Rehabilitation—US 2022/0111257.

Each of the above is incorporated herein by reference in its entirety.

To collect data, subjects or users may walk on a pressure-sensitive treadmill while simultaneously carrying two smartphones, one in each side pocket on their pants (or one strapped to each leg using a special pouch, for users not wearing pants with proper pockets). The smartphones may run the commercially available OneStep physical therapy application and record the subject's walk, using the IMU sensors within the smartphones and a OneStep app, where OneStep is an FDA-listed medical app, downloadable from GooglePlay, that uses smartphone motion sensors to provide immediate, clinically-validated feedback on gait inter alia.

The treadmill, using a force plate beneath its belt, measures force and moment data many times per second. Three specific force measurements may be used in the subsequent process: Fz, which is the total force in the downward direction applied to the treadmill belt at each timepoint; and/or COPx and/or COPY, the coordinates of the center of pressure on the treadmill belt in the x- and y-axis (the two axes of the transverse plane of the subject).

Gold standard COP measurement may occur in operation 1010a of FIG. 9.

When generating a COP gold standard measurement, e.g., in operation 1010a of FIG. 9, the MoTek treadmill, available from motekmedical.com, or the Zeno walk mat, or any force plate system may, by way of non-limiting example, be used (aka ground force system). Manufacturer's instructions may be followed to install and calibrate the system for any subject being measured. The system is then ready to record the subject's kinetics and ground forces, and hence, recording IMU data may commence using smartphones placed in various bodily positions (such as pants' pockets). Subjects are instructed to perform different gait patterns (such as, say, all or any subset of walking with long/short steps, long horizontal distance between feet, different back postures, different postures of the feet while touching the ground, different speeds, different cadence, different flexion of the hip joint, no flexion of the knee), while the subject's motion is recorded on the smartphones and the ground force system. The system control software is marked when new instructions are provided and the gait pattern changes. This stores timestamps of changes in gait patterns over the recording. Eventually, COP recording and IMU recording of the same activity is obtained, with additional timestamp markers of change of gait.

### Measurement Synchronization e.g., for Operation 1010b of FIG. 9

In order to provide models, e.g., for training purposes, with pairs of IMU-repetitive motion representation, typically generated by segmentation into IMU cycles or gait cycles or strides, and their corresponding lower body kinematic data, the motion capture sensors and the IMU system are typically synchronized.

Any suitable method may be employed to synchronize the systems, such as calibration of the gold-standard ground force system's and IMU's respective internal clocks with each other, or figuring out the offset between the gold-standard and IMU, if the reference system's (e.g., gold standard system's) internal clock is accessible. Another alternative, e.g., if the internal clock is not accessible, is a high-frequency (e.g., FPS (frames per second) higher than 100) video that captures the internal clock presented by the IMU measurement device (e.g., the IMU is installed in a smartphone, and the IMU's measurement clock is presented on the smartphone's screen), the events being measured by the reference system. By detecting the first temporal event measured by the reference system in the video, the system's clock offset, which refers to the video time, may be found, and the offset of the video with the IMU device may be found by extracting the IMU device clock's timestamp shown on the video.

According to certain embodiments, after completion of the subject's walk on the treadmill, the treadmill's software (e.g.,
https://knowledge.hocoma.com/wp-content/uploads/2019/03/C-Mill-Therapy-guide v8.0.pdf) provides both a file of the timepoint-by-timepoint force data as described above, as well as a file of the times of each gait event (heel strike and toe-off), as determined by the shifts in force and center of pressure. The system may then temporally align the treadmill data and the smartphone gait data in the following way. The system may then take the time points of all heel strikes in the primary leg (that is, the leg on which the phone was attached to/in the pocket for the smartphone recording), from both the treadmill and smartphone data. The system may then compute the cross-correlation between the two arrays of timepoints. The shift which generates the minimum cross-correlation corresponds to the proper alignment of the smartphone and treadmill data, and that shift may be subtracted from the smartphone data time to align the two measurements' reference timeframe.

After syncing the signals in operation 1010b of FIG. 9, the data is segmented in operation 1010c. In operation 1010c, typically, the raw IMU data representing repetitive motion is segmented into intervals aka strides, e.g., as described in co-pending patent documents referenced elsewhere herein and incorporated herein by reference in their entirety, then the segmentation may be applied to the COP signal, to create segments of COP data. The segmented COP data is the path of the center of pressure in two dimensions over the gait cycle, which may be represented in the form of a “butterfly” diagram.

### Replacing the Segmented COP Labels With Any Function of Segmented COP May Occur in Operation 10d

Other parameters that can be extracted are the maximum, average, and sums of forces over time at various gait stages, such as single support of each leg, double support, and the time surrounding each gait event (heel strike and toe-off). These parameters can also be normalized by the weight of each subject, since these force-based parameters typically depend linearly on the weight of the subject; this normalization can help extract more clinically useful information from these parameters.

### Generating an ML Model (e.g., for Operation 1020 of FIG. 8)

To generate these COP force-based parameters from the IMU data, a machine learning framework may be used.

For example, for individual kinetic parameters (e.g., maximum loading force during heel strike), a deep learning model may be trained, using the IMU data as input and the associated parameter values as labels (after splitting the data into training and validation subsets). In this way, a model may be created that takes an IMU data as input and predicts the parameter value for the specific parameter that the model was trained on.

For the “butterfly” of the center of pressure, a more complex model may be employed, as the butterfly represents a three-dimensional measurement over time (two for position, and the third one for the force magnitude), rather than a single scalar value associated with the IMU segmented data. To train that model, encoder-decoder architecture may be employed, e.g., as described in https://d21.ai/chapter recurrent-modern/encoder-decoder.html

Distance/s between the generated butterfly and real butterfly may (e.g., as described above) be measured and optimized e.g. a Euclidean distance and a correlation distance. The first measures the differences between the center of pressure paths in magnitude, and the second measures the difference in contour. Computing the loss using a combination of plural methods, e.g., both of the above distance quantification methods typically leads to better performance than using just one distance metric.

### Generating an ML Model (e.g., for Operation 1020 of FIG. 8)

Methods for ML model generation, which may be used to implement operation 20 thereby to yield a trained model which may be used, for example, in operations 40 and 50, are now described.

A machine learning model may be used to reconstruct kinematics from IMU data. The model typically has an encoder part (e.g. as described in Wikipedia's entry on “autoencoder”) that takes kinematics input and creates a compressed code representation of the original kinematics input, typically using a process known as embedding or encoding. The encoder is paired with a decoder that takes compressed codes as input and extracts kinematics data from the decoder. This coupling is used as an auto-encoder and is trained together in order for the decoder to extract the same kinematic data used as input for the encoder.

The model typically has a second encoder part (aka IMU encoder) which may be programmed e.g. as described in https://d21.ai/chapter recurrent-modern/encoder-decoder.html and which may be used to take IMU input (IMU repetitive segment representation) and compress the IMU input as code representation. The IMU encoder may be paired with the kinematic encoder to create the same representation for IMU data as its counterpart creates for the matching kinematic data. In this way the same decoder can be used to extract the kinematic information from the code created by IMU encoder, while the kinematic autoencoder ensures it has the same kinematic data as the original.

Given an IMU input, the model uses the IMU encoder followed by the kinematic decoder and reconstructs the corresponding kinematic data (e.g. in operation 40). This may be referred to as the generative model.

It is appreciated that any suitable known method may be used to perform embedding or encoding on the input before the embedded vector is decoded to the COP space (e.g., to the cycle of 3X100 for x, y,force).

Validation in the training process may be achieved by comparing the kinematic output with the kinematic data recorded alongside the IMU data e.g. using forward kinematics as described below.

### Validation Through Forward Kinematics (Operation 2050 in FIG. 13)

Validation after the training process (e.g., in operation 2050) is done using forward kinematics, as the kinematic data is not captured directly with the IMU and therefore is missing in post-training usage. Forward kinematics typically comprises the use of analytic equations by a hardware processor, to compute position and orientation of the end-effector from its joints, and links parameter values. The equations typically have a closed form comprising a chain of rigid body transformations, such as rotations and translations of the links and joints of the body, and therefore is computable, given kinematic input.

The model has a forward kinematics portion that is applied on the reconstructed kinematic output to yield the IMU data back, since the IMU data is the trajectory (position/acceleration and orientation) of the IMU measurement device approximately. This data is then compared with the original IMU input to validate the kinematic reconstruction process. The training process (operation 20) uses the training data to minimize the gap or distance between the original IMU data and the reconstructed IMU data. Similarities between the input and results may be quantified by measuring both the correlation in signal form and magnitude, e.g., as described above.

Distance may be measured e.g., in degrees MSE (mean square error) across all channels. It turns out that similar IMU data, below 1.7 degrees MSE, corresponds with similar kinematics data, below 2.5 degrees MSE, at 95 percentile, and therefore can be used as a threshold for a validator for a kinematic reconstruction model.

It is appreciated that typically, both input and output are cyclic. Typically, the system input comprises 100 (say) IMU values and system output comprises the same number (typically) of inertial or non-IMU values.

According to one embodiment, 100 (say) ML-models are trained, each inputting and outputting one value. However, according to other embodiments, a single ML model is trained, rather than 100 (say) such models, so that the single model may learn from cross-interactions between the 100 IMU values. For example, generating the COP at point 87 (from among 100 points along the gait cycle) makes use of IMU data not just at point 87, but also others from among the other 99 (say) points, and typically from the whole cycle of 1-100 points or values.

The method of FIG. 6 may be implemented in accordance with any of the operations in co-pending Approximating Motion Capture of Plural Body Portions using a Single IMU Device (Publication number: 20230137198 and/or may include all or any subset of the following:

Stage a (aka Stage 30c-a)—Adjustment and Standardization of Repetitions

For each of the data segments of the repetitions, perform all or any subset of the following operations, suitably ordered e.g., as shown:

a-1: Ignoring the offset of the yaw channel—the north is irrelevant to defining the frame of reference as defined above, and similarly any other offset of the yaw, since given a motion record of someone walking, the direction of the walk is not relevant, and it is desirable for the representation of that activity to be invariant of the direction. Hence, the average yaw may be subtracted for each repetition, whether the data is calibrated to the north, or showing a false reference of the north. Other techniques may also be used, such as splitting the yaw channel into two-one channel for the azimuth (a smoothed version of the yaw represents the macro changes of the yaw), and the other channel for subtraction of the azimuth from the yaw, which represents the micro changes of the yaw.

A-2. Embed data in uncalibrated reference frame e.g., as described elsewhere herein. Rotating (using conventional rotation arithmetic) the acceleration channel with the corresponding orientation from the orientation channel produces acceleration in the global reference frame of earth (X=north, Y=west, Z=towards the sky). Multiplying the orientation channel with their inverse mean of orientation on the right produces rotations that are relative to the mean orientation of the segment, aka the relative orientations. By rotating the acceleration and producing the relative orientations, the relation between the data and the orientation of the measurement system (the IMU device) over the body is eliminated, and what remains is accelerations and relative rotations in the uncalibrated reference frame, e.g., a reference frame in which the X is, say, the north, rather than the course of movement. Since the yaw channel is neutralized in operation a-1, X is an arbitrary north, rather than the geographical north. Typically, neither the geographical north nor the arbitrary north are helpful for defining the course of movement, hence the method may neutralize the north to render the remainder of the process independent of the course of motion in the global reference frame.

A-3. Displacement and orientation adjustment-typically, the data is presented as displacement, although the data could also be presented as accelerations. The displacement is smoother, enables understanding of the motion better for human eyes, and preserves the information of the original data, which may be reversed by derivating or computing a derivative of the displacement. The reference frame moves with the movement, meaning that the displacement over the data segment may be assumed to be 0 which determines the initial velocity (the velocity may also be assumed not to change over the data segment, meaning that there are no accelerations between repetitions, which turns out to be a reasonable assumption for long continuous repetitions. In addition, the effect on the data is minor, which is subtraction of the average acceleration over the data segment (the average acceleration may be kept). This assumption yields benefits: the data may be fixed due to noise or biases of the sensors which are reasonable, and the displacement is not only closed (as it starts and ends at the same displacement) but is also smooth, since the velocity is the same at the beginning and at the end. This makes any shift of data from the end of the segment to its beginning yield the same displacement, yielding invariance to the determination of phase 0, the starting point of the repetitions' segments. The orientation of the starting point and the last point may be constrained to be the same.

Thus, according to certain embodiments, the average acceleration is subtracted from the accelerations and integrated over the segment to get velocities; then the average velocity is subtracted from the velocities and integrated over the segment to get the displacement. Orientations are handled after the reference frame has been fixed.

a-4. Interpolation-interpolate the displacement and the orientation of each segment to have (say) 100 equal time units, to ensure a standard structure for each repetition (with an equal number of time units).

Stage b (aka Stage 30c-b)—Determination of the course of movement

Rotate the reference frame such that X and Z axes will be calibrated, where the course of movement is the X-axis. This rotation depends on the orientation of the measurement system referred to the motion itself; typically, all of the repetitions' representations at this stage are embedded in coherent reference frames, which may need to be calibrated with the same rotation. This rotation may be estimated using any suitable technique, such as applying principal component analysis (PCA) on the projections of either the displacements or the orientations on the horizontal plane (which is known, since the vertical axis is known). The most significant component of the displacement may imply the direction of the movement because this is the direction in which the most significant changes typically occur. Similarly, the most significant component of the orientations represented as rotation vectors may imply the Z-axis around which the most significant changes of orientations typically occur (e.g., in walking measured from the thigh (e.g., if the IMU is adjacent to the end-user's thigh), the most significant component of the orientations over the horizontal plane is the hip flexion-extension axis). Either of these (the most significant component of the displacement, or the most significant changes of orientations) may be used to rotate the reference frame. Eventually, this yields all of the data represented in a calibrated frame of reference. The orientations may then be converted to Euler representation in the order of Z-X-Y, and the average angle velocities of each orientation channel may be subtracted to verify each of them starts and ends at the same angle, which yields invariance to the initial point of the segmentations e.g., as described above.

It is appreciated that the above process may be used to implement stage 10-1-b of FIG. 7, and may also be performed stand-alone.

Stage c (aka Stage 30c-c)—Aggregation of the Repetitions

The repetitions' representations are similar to each other, even initially, and, typically, after standardization of their structure and elimination of irrelevant attributes of measurement and analysis, the repetitions' representations become even more similar. Some tasks may use one aggregated representation of the motion, rather than on each of the repetitions, for example, recognition of the activity being performed by the subject and the position of the measurement device e.g., IMU as well. For this kind of task, the aggregated form of the repetitions' representations may be employed. This process may, for example, comprise naive averaging each of the channels over every one of 100 (say) time-units, or by dynamic time warping (DTW) techniques. In practice, the naive aggregation method, while less sophisticated, nonetheless works well.

It is appreciated that, alternatively, the data segmentations may be taken as-is, and stages a, b, c may be omitted or may be implemented in any suitable manner other than as above.

It is appreciated that any operation or embodiment herein may be combined with any operation or embodiment described in the co-pending patent document entitled “Approximating Motion Capture of Plural Body Portions using a Single IMU Device” and incorporated herein by reference in its entirety which describes capturing motion of a moving body, including an IMU interface to receive IMU measurements from IMU/s worn on the body; and a processor to derive, from the IMU measurements, a motion capture approximation output including a trajectory, which describes a body portion's motion during a cycle of repetitive motion, yielding a trajectory set including B body portion trajectories, wherein, to derive the trajectory set, the processor uses generative adversarial networks including one network trained to determine physical feasibility of candidate body portion trajectory/ies for body portion/s, from among multiple candidate body portion trajectories for the specific body portion; and another network trained to determine how well candidate body portion trajectory/ies fit/s the IMU measurements.

It is appreciated that any operation or embodiment herein may be combined with any operation or embodiment described in the co-pending patent document entitled “System, Method, and Computer Program Product for Detecting a Mobile Phone User's Risky Medical Condition” available under USPTO Publication number: 20200375544 and incorporated herein by reference in its entirety, which describes a stroke detection system operative to detect strokes suffered by mobile communication device users, the system comprising a hardware processor operative in conjunction with a mobile communication device having at least one built-in sensor; the hardware processor being configured to, repeatedly and without being activated by the device's user, compare data derived from said at least one sensor to at least one baseline value for at least one indicator of user well-being, stored in memory accessible to the processor and/or make a stroke risk level evaluation; and/or perform at least one action if and only if the stroke risk level is over a threshold.

It is appreciated that any operation or embodiment herein may be combined with any operation or embodiment described in the co-pending patent document entitled “System, Method, and Computer Program Product for Assessment of a User's Gait” available under USPTO Publication number 20200289027 and incorporated herein by reference in its entirety, which describes a gait monitoring system operative to monitor gait of an end-user bearing a wearable device equipped with at least one magneto-inertial sensor, the system comprising a processor configured to receive raw sensor data from the wearable device's at least one magneto-inertial sensor to extract situational data from the raw sensor data, the situational data including at least the device's bodily position relative to the end-user, to determine a gait analysis process which yields at least one parameter characterizing the end-user's gait, depending at least on the device's bodily position as extracted, and to compute, and generate an output indication of, the at least one parameter characterizing the end-user's gait, by running the gait analysis process as selected.

It is appreciated that terminology such as “mandatory”, “required”, “need” and “must” refer to implementation choices made within the context of a particular implementation or application described herewithin for clarity, and are not intended to be limiting, since, in an alternative implementation, the same elements might be defined as not mandatory and not required. or might even be eliminated altogether.

Components described herein as software may, alternatively, be implemented wholly or partly in hardware and/or firmware, if desired, using conventional techniques, and vice-versa. Each module or component or processor may be centralized in a single physical location or physical device or distributed over several physical locations or physical devices.

Included in the scope of the present disclosure, inter alia, are electromagnetic signals in accordance with the description herein. These may carry computer-readable instructions for performing any or all of the operations of any of the methods shown and described herein, in any suitable order, including simultaneous performance of suitable groups of operations, as appropriate. Included in the scope of the present disclosure, inter alia, are machine-readable instructions for performing any or all of the operations of any of the methods shown and described herein, in any suitable order; program storage devices readable by machine, tangibly embodying a program of instructions executable by the machine to perform any or all of the operations of any of the methods shown and described herein, in any suitable order, i.e., not necessarily as shown, including performing various operations in parallel or concurrently, rather than sequentially, as shown; a computer program product comprising a computer useable medium having computer readable program code, such as executable code, having embodied therein, and/or including computer readable program code for performing, any or all of the operations of any of the methods shown and described herein, in any suitable order; any technical effects brought about by any or all of the operations of any of the methods shown and described herein, when performed in any suitable order; any suitable apparatus or device or combination of such, programmed to perform, alone or in combination, any or all of the operations of any of the methods shown and described herein, in any suitable order; electronic devices each including at least one processor and/or cooperating input device and/or output device and operative to perform, e.g., in software, any operations shown and described herein; information storage devices or physical records, such as disks or hard drives, causing at least one computer or other device to be configured so as to carry out any or all of the operations of any of the methods shown and described herein, in any suitable order; at least one program pre-stored e.g. in memory or on an information network such as the Internet, before or after being downloaded, which embodies any or all of the operations of any of the methods shown and described herein, in any suitable order, and the method of uploading or downloading such, and a system including server/s and/or client/s for using such; at least one processor configured to perform any combination of the described operations or to execute any combination of the described modules; and hardware which performs any or all of the operations of any of the methods shown and described herein, in any suitable order, either alone or in conjunction with software. Any computer-readable or machine-readable media described herein is intended to include non-transitory computer-or machine-readable media.

Any computations or other forms of analysis described herein may be performed by a suitable computerized method. Any operation or functionality described herein may be wholly or partially computer-implemented, e.g., by one or more processors. The invention shown and described herein may include (a) using a computerized method to identify a solution to any of the problems or for any of the objectives described herein, the solution optionally including at least one of a decision, an action, a product, a service or any other information described herein that impacts, in a positive manner, a problem or objectives described herein; and (b) outputting the solution.

The system may, if desired, be implemented as a network-e.g., web-based system employing software, computers, routers, and telecommunications equipment, as appropriate.

Any suitable deployment may be employed to provide functionalities, e.g., software functionalities shown and described herein. For example, a server may store certain applications, for download to clients, which are executed at the client side, the server side serving only as a storehouse. Any or all functionalities, e.g., software functionalities shown and described herein, may be deployed in a cloud environment. Clients, e.g., mobile communication devices such as smartphones, may be operatively associated with, but external to the cloud.

The scope of the present invention is not limited to structures and functions specifically described herein and is also intended to include devices which have the capacity to yield a structure, or perform a function, described herein, such that even though users of the device may not use the capacity, they are, if they so desire, able to modify the device to obtain the structure or function.

Any “if-then” logic described herein is intended to include embodiments in which a processor is programmed to repeatedly determine whether condition x, which is sometimes true and sometimes false, is currently true or false, and to perform y each time x is determined to be true, thereby to yield a processor which performs y at least once, typically on an “if and only if” basis, e.g., triggered only by determinations that x is true, and never by determinations that x is false.

Any determination of a state or condition described herein, and/or other data generated herein, may be harnessed for any suitable technical effect. For example, the determination may be transmitted or fed to any suitable hardware, firmware, or software module, which is known or which is described herein to have capabilities to perform a technical operation responsive to the state or condition. The technical operation may, for example, comprise changing the state or condition, or may more generally cause any outcome which is technically advantageous, given the state or condition or data, and/or may prevent at least one outcome which is disadvantageous, given the state or condition or data. Alternatively or in addition, an alert may be provided to an appropriate human operator or to an appropriate external system.

Features of the present invention, including operations which are described in the context of separate embodiments, may also be provided in combination in a single embodiment. For example, a system embodiment is intended to include a corresponding process embodiment, and vice versa. Also, each system embodiment is intended to include a server-centered “view” or client centered “view”, or “view” from any other node of the system, of the entire functionality of the system, computer-readable medium, apparatus, including only those functionalities performed at that server or client or node. Features may also be combined with features known in the art, and particularly, although not limited to those described in the Background section or in publications mentioned therein.

Conversely, features of the invention, including operations, which are described for brevity in the context of a single embodiment or in a certain order, may be provided separately or in any suitable sub-combination, including with features known in the art (particularly although not limited to those described in the Background section or in publications mentioned therein) or in a different order. “e.g.” is used herein in the sense of a specific example which is not intended to be limiting. Each method may comprise all or any subset of the operations illustrated or described, suitably ordered e.g. as illustrated or described herein.

Devices, apparatus or systems shown coupled in any of the drawings may in fact be integrated into a single platform in certain embodiments, or may be coupled via any appropriate wired or wireless coupling, such as but not limited to optical fiber, Ethernet, Wireless LAN, HomePNA, power line communication, cell phone, Smart Phone (e.g. iPhone), Tablet, Laptop, PDA, Blackberry GPRS, Satellite including GPS, or other mobile delivery. It is appreciated that in the description and drawings shown and described herein, functionalities described or illustrated as systems and sub-units thereof can also be provided as methods and operations therewithin, and functionalities described or illustrated as methods and operations therewithin can also be provided as systems and sub-units thereof. The scale used to illustrate various elements in the drawings is merely exemplary and/or appropriate for clarity of presentation, and is not intended to be limiting.

Any suitable communication may be employed between separate units herein, e.g., wired data communication and/or in short-range radio communication with sensors such as cameras e.g., via Wifi, Bluetooth, or Zigbee.

It is appreciated that implementation via a cellular app as described herein is but an example, and, instead, embodiments of the present invention may be implemented, say, as a smartphone SDK; as a hardware component; as an STK application, or as suitable combinations of any of the above.

Any processing functionality illustrated (or described herein) may be executed by any device having a processor, such as but not limited to a mobile telephone, set-top-box, TV, remote desktop computer, game console, tablet, mobile e.g. laptop or other computer terminal, embedded remote unit, which may either be networked itself (may itself be a node in a conventional communication network e.g.) or may be conventionally tethered to a networked device (to a device which is a node in a conventional communication network, or is tethered directly or indirectly/ultimately to such a node).

Any operation or characteristic described herein may be performed by another actor outside the scope of the patent application and the description is intended to include apparatus whether hardware, firmware or software which is configured to perform, enable, or facilitate that operation or to enable, facilitate, or provide that characteristic.

The terms processor or controller or module or logic as used herein are intended to include hardware such as computer microprocessors or hardware processors, which typically have digital memory and processing capacity, such as those available from, say Intel and Advanced Micro Devices (AMD). Any operation or functionality or computation or logic described herein may be implemented entirely or in any part on any suitable circuitry including any such computer microprocessor/s as well as in firmware or in hardware or any combination thereof.

It is appreciated that elements illustrated in more than one drawing, and/or elements in the written description, may still be combined into a single embodiment, except if otherwise specifically clarified herewithin. Any of the systems shown and described herein may be used to implement or may be combined with, any of the operations or methods shown and described herein.

It is appreciated that any features, properties, logic, modules, blocks, operations, or functionalities described herein which are, for clarity, described in the context of separate embodiments, may also be provided in combination in a single embodiment, except where the specification or general knowledge specifically indicates that certain teachings are mutually contradictory and cannot be combined. Any of the systems shown and described herein may be used to implement or may be combined with, any of the operations or methods shown and described herein.

Conversely, any modules, blocks, operations or functionalities described herein, which are, for brevity, described in the context of a single embodiment, may also be provided separately or in any suitable sub-combination, including with features known in the art. Each element, e.g., operation described herein may have all characteristics and attributes described or illustrated herein, or, according to other embodiments, may have any subset of the characteristics or attributes described herein.

It is appreciated that apps implementing any functionality herein may include a cell app, mobile app, computer app, or any other application software. Any application may be bundled with a computer and its system software, or published separately. The term “phone” and similar used herein is not intended to be limiting and may be replaced or augmented by any device having a processor, such as but not limited to a mobile telephone, or also set-top-box, TV, remote desktop computer, game console, tablet, mobile, e.g., laptop or other computer terminal, embedded remote unit, which may either be networked itself (may itself be a node in a conventional communication network e.g.) or may be conventionally tethered to a networked device (to a device which is a node in a conventional communication network or is tethered directly or indirectly/ultimately to such a node). Thus, the computing device may even be disconnected from e.g., WiFi, Bluetooth, etc., but may be tethered directly or ultimately to a networked device.

References herein to “said (or the) element x” having certain (e.g., functional or relational) limitations/characteristics, are not intended to imply that a single instance of element x is necessarily characterized by all the limitations/characteristics. Instead, “said (or the) element x” having certain (e.g. functional or relational) limitations/characteristics is intended to include both (a) an embodiment in which a single instance of element x is characterized by all of the limitations/characteristics and (b) embodiments in which plural instances of element x are provided, and each of the limitations/characteristics is satisfied by at least one instance of element x, but no single instance of element x satisfies all limitations/characteristics. For example, each time L limitations/characteristics are ascribed to “said” or “the” element X in the specification or claims (e.g. to “said processor” or “the processor”), this is intended to include an embodiment in which L instances of element X are provided, which respectively satisfy the L limitations/characteristics, each of the L instances of element X satisfying an individual one of the L limitations/characteristics. The plural instances of element x need not be identical. For example, if element x is a hardware processor, there may be different instances of x, each programmed for different functions and/or having different hardware configurations (e.g., there may be 3 instances of x: two Intel processors of different models, and one AMD processor).

1. A gait analysis method comprising:
providing a data flow generated by at least one inertial sensor borne by an end-user who is ambulating, thereby to define a data flow which describes the end-user's gait, and
providing a hardware processor configured to derive, from the data flow which describes the end-user's gait, center of pressure (COP) trajectory data characterizing the end-user.

2. A gait analysis method comprising:
providing a data flow generated by at least one inertial sensor borne by an end-user who is ambulating, thereby to define a data flow which describes the end-user's gait,
providing a hardware processor configured to derive, from the data flow which describes the end-user's gait, kinematic data characterizing the end-user;
estimating validity of using the inertial sensor to estimate kinematic data and
generating an output indication of the user's kinematics trajectory data only when said validity is over-threshold.

3. A method according to claim 1 wherein the end-user bears a mobile phone having an integral accelerometer and wherein the single sensor comprises the mobile phone's integral accelerometer.

4. A method according to claim 1 wherein a machine-learning model resides in the processor and wherein the machine-learning model is trained, using training data including:
plural data flows each generated by a single inertial sensor borne by a user, thereby to define plural users, and
plural labels comprising center of pressure (COP) trajectory data regarding the plural users respectively which was collected using multiple sensors to sense each of the users' gaits.

5. A method according to claim 1 wherein the center of pressure (COP) trajectory data characterizing the end-user comprises a prototypical center of pressure (COP) trajectory which the end-user's COP approximately follows each time the end-user proceeds through her/his gait cycle.

6. A method according to claim 1 wherein said at least one inertial sensor comprises at least one accelerometer.

7. A method according to claim 1 wherein said at least one inertial sensor comprises hardware within a mobile device e.g., phone.

8. A method according to claim 1 wherein said at least one inertial sensor comprises a single sensor.

9. A method according to claim 1 wherein the trajectory data which the hardware processor is configured to derive, comprises trajectory/ies of lower body joint angles aka joint kinematics data characterizing the end-user.

10. A method according to claim 1 wherein the trajectory data which the hardware processor is configured to derive, comprises center of pressure (COP) trajectory data characterizing the end-user.

11. A method according to claim 10 wherein the center of pressure (COP) trajectory data characterizing the end-user is presented to at least one user as a butterfly diagram.

12. A method according to claim 2 wherein the at least one inertial sensor comprises a single inertial sensor.

13. A gait analysis system comprising:
a hardware processor configured for providing a data flow generated by at least one inertial sensor borne by an end-user who is ambulating, thereby to define a data flow which describes the end-user's gait, and
a hardware processor configured to derive, from the data flow which describes the end-user's gait, center of pressure (COP) trajectory data characterizing the end-user.
